(19) United States
(12) Patent Application Publication
(10) Pub. No.: US 2014/0051771 A1
(43) Pub. Date: Feb. 20, 2014

(54) Biomedical Devices Comprising Molded Polyethylene Components

(71) Applicant: Ticona LLC, Florence, KY (US)

(72) Inventors: Julia Hufen, Rheinberg (DE); Anthony Verrocchi, Covington, KY (US); Rainer Walkenhorst, Melle (DE)

(73) Assignee: Ticona LLC, Florence, KY (US)

(21) Appl. No.: 13/964,884

(22) Filed: Aug. 12, 2013

Related U.S. Application Data
(60) Provisional application No. 61/787,237, filed on Mar. 15, 2013.
(60) Provisional application No. 61/684,947, filed on Aug. 20, 2012.

(51) Int. Cl. C08F 10/02; B29D 23/00; A61N 1/05

(52) U.S. Cl. CPC C08F 10/02; A61N 1/0551; B29D 23/00
USPC 514/772.4; 526/352; 526/348.6; 526/348; 525/333.7; 521/143; 524/585; 525/55; 525/185; 525/190; 525/240; 525/70; 525/232; 525/222; 525/227; 525/238; 607/116; 607/119; 607/137; 428/523; 428/516; 428/461; 428/451; 428/36.9; 53/441; 264/250

(57) ABSTRACT

A molded polyethylene component that comprises polyethylene having a melt flow index of about 3.5 g/10 min or greater as measured by ASTM D1238 at 190° C./21.5 kg weight and having an ash content of about 500 ppm or less may be useful biomedical devices, including kits and methods relating thereto.

### BACKGROUND

The present invention relates to molded polyethylene components for use in conjunction with biomedical devices, including methods relating thereto.

Polyethylene is often used in conjunction with biomedical devices because of, inter alia, its chemical resistance, biocompatibility, low specific gravity relative to metals, and wear resistance. Typically, polyethylene, having a purity sufficient for use in biological environments (e.g., minimal concentration of residual catalyst), has a low melt flow index, and in some instances essentially no melt flow index. Due to the hardness of low melt flow index polyethylene, production of biomedical devices and components therefrom is typically achieved through machining methods. While machining fabrication methods may allow for high precision components capable of meeting tight tolerances, these methods generally have a low throughput and can produce significant waste of the polyethylene material, both of which increase the cost of manufacturing. Further, machine fabrication can require large capital investments in equipment. Accordingly, methods and compositions that enable higher throughput manufacturing, like molding, may be desirable in the production of polyethylene-based biomedical device components.

### SUMMARY OF THE INVENTION

The present invention relates to molded polyethylene components for use in conjunction with biomedical devices, including methods relating thereto.

In one embodiment, the present invention provides for a biomedical device that comprises a molded polyethylene component that comprises a polyethylene composition that comprises polyethylene having a melt flow index of about 3.5 g/10 min or greater as measured by ASTM D1238 at 190° C./21.5 kg weight and having an ash content of about 500 ppm or less.

In another embodiment, the present invention provides for a biomedical device that comprises a conduit that comprises at least one wall defining an internal volume, the at least one wall comprising polyethylene having a melt flow index of about 3.5 g/10 min or greater as measured by ASTM D1238 at 190° C./21.5 kg weight and having an ash content of about 500 ppm or less.

In yet another embodiment, the present invention provides for a method that comprises providing a polyethylene composition melt that comprises polyethylene having a melt flow index of about 3.5 g/10 min or greater as measured by ASTM D1238 at 190° C./21.5 kg weight and having an ash content of about 500 ppm or less; and extruding the polyethylene composition melt to form a molded polyethylene component of a biomedical device.

In another embodiment, the present invention provides for a method that comprises providing a polyethylene composition melt that comprises polyethylene having a melt flow index of about 3.5 g/10 min or greater as measured by ASTM D1238 at 190° C./21.5 kg weight and having an ash content of about 500 ppm or less; and injection molding the polyethylene composition melt to form a molded polyethylene component of a biomedical device.

In yet another embodiment, the present invention provides for a method that comprises providing a component of a biomedical device; and substantially encasing the component in polyethylene having a melt flow index of about 3.5 g/10 min or greater as measured by ASTM D1238 at 190° C./21.5 kg weight and having an ash content of about 500 ppm or less.

In yet another embodiment, the present invention provides for a method that comprises polymerizing at least one olefin in the presence of a catalyst so as to form polyethylene having a melt flow index of about 3.5 g/10 min or greater as measured by ASTM D1238 at 190° C./21.5 kg weight, the at least one olefin being selected from the group consisting of ethylene, propylene, and/or butylene, and any combination thereof; and treating the polyethylene to yield a treated polyethylene having an ash content of about 500 ppm or less.

In another embodiment, the present invention provides for a method that comprises providing a sheet comprising a polyethylene composition that comprises polyethylene having a melt flow index of about 3.5 g/10 min or greater as measured by ASTM D1238 at 190° C./21.5 kg weight and having an ash content of about 500 ppm or less; and thermoforming the sheet into a molded polyethylene component of a biomedical device.

In yet another embodiment, the present invention provides for a method that comprises providing a sheet comprising a polyethylene composition that comprises polyethylene having a melt flow index of about 3.5 g/10 min or greater as measured by ASTM D1238 at 190° C./21.5 kg weight and having an ash content of about 500 ppm or less; and shrink wrapping the sheet onto a portion of a biomedical device or component thereof.

The features and advantages of the present invention will be readily apparent to those skilled in the art upon a reading of the description of the preferred embodiments that follows.

### DETAILED DESCRIPTION

The present invention relates to molded polyethylene components for use in conjunction with biomedical devices, including methods relating thereto. In some instances, the molded polyethylene components may be biomedical devices themselves.

The molded polyethylene components of the present invention for use in conjunction with biomedical devices may, in some embodiments, comprise polyethylene compositions with high purity that is melt flowable, thereby enabling the molded polyethylene components to be produced via molding and/or extruding methods. The ability to form a polyethylene component via these methods may advantageously enable faster production of high precision components as compared to machining methods. Molding method may also enable the production of complex and smaller components that are time and cost prohibitive if produced by machining methods. Further, the waste associated with manufacturing may be reduced as compared to machining methods. Accordingly, for at least these reasons, the ability to mold and/or extrude polyethylene components of the present invention for use in conjunction with biomedical devices as described herein may, in turn, reduce manufacturing costs, and consequently consumer costs, which especially in the biomedical sector may be especially advantageous.

It should be noted that when “about” is provided at the beginning of a numerical list, “about” modifies each number of the numerical list. It should be noted that in some numerical listings of ranges, some lower limits listed may be greater than some upper limits listed. One skilled in the art will recognize that the selected subset will require the selection of an upper limit in excess of the selected lower limit.

In some embodiments, a biomedical device may comprise a molded polyethylene component of the present invention. A molded polyethylene component of the present invention may, in some embodiments, comprise a polyethylene composition that comprises a high-purity, melt-flowable polyethylene. It should be noted that the term “molded polyethylene component” is used herein for clarity and should not be seen as limiting as to how the polyethylene component is produced. As used herein, the terms “high-purity, melt-flowable polyethylene” and “HPMF polyethylene” refers to a composition that comprises polyethylene having a melt flow index of about 3.5 g/10 min or greater as measured by ASTM D1238 at 190° C./21.5 kg weight and having an ash content of about 500 ppm or less. It should be noted that as used herein “polyethylene” encompasses copolymers of ethylene with other olefinic monomers, e.g., propylene, butylene, and the like, including mixtures and blend polymers thereof.

In some embodiments, the HPMF polyethylene described herein of the molded polyethylene component of the present invention may have a melt flow index of about 3.5 g/10 min or greater, about 5 g/10 min or greater, or about 7 g/10 min or greater as measured by ASTM D1238 at 190° C./21.5 kg weight. In some embodiments, the HPMF polyethylene described herein of the molded polyethylene component of the present invention may have a melt flow index ranging from a lower limit of about 3.5 g/10 min, 5 g/10 min, or 10 g/10 min to an upper limit of about 30 g/10 min, 25 g/10 min, 20 g/10 min, or 15 g/10 min as measured by ASTM D1238 at 190° C./21.5 kg weight.

In some embodiments, the HPMF polyethylene described herein of the molded polyethylene component of the present invention may have an ash content of about 500 ppm or less, about 250 ppm or less, about 100 ppm or less, 50 ppm or less, or 10 ppm or less. In some embodiments, the HPMF polyethylene described herein of the molded polyethylene component of the present invention may have an ash content ranging from a lower limit of about 0.1 ppm, 1 ppm, 10 ppm, 25 ppm, 50 ppm, 75 ppm, 100 ppm, or 150 ppm to an upper limit of about 500 ppm, 400 ppm, 300 ppm, 250 ppm, 200 ppm, 150 ppm, 100 ppm, 50 ppm, or 10 ppm, and wherein the ash content may range from any lower limit to any upper limit and encompass any subset therebetween.

In some embodiments, the HPMF polyethylene described herein of the molded polyethylene component of the present invention may be compliant with USP Class IV and/or ISO 10993 standards at the filing date of this application.

In some embodiments, the HPMF polyethylene described herein of the molded polyethylene component of the present invention may be further characterized as having a specific gravity of about 0.93 g/cm³ to about 0.97 g/cm³.

In some embodiments, the HPMF polyethylene described herein of the molded polyethylene component of the present invention may have an average molecular weight ranging from a lower limit of about 30,000 g/mol, 50,000 g/mol, 100,000 g/mol, 250,000 g/mol, or 500,000 g/mol to an upper limit of about 2,500,000 g/mol, 2,000,000 g/mol, 1,000,000 g/mol, or 500,000 g/mol, and wherein the average molecular weight may range from any lower limit to any upper limit and encompass any range therebetween. Methods suitable for measuring the molecular weight of the HPMF polyethylene described herein include ASTM D4020.

In some embodiments, the HPMF polyethylene described herein of the molded polyethylene component of the present invention may have a molecular weight distribution that is multimodal (i.e., having more than one local maxima, e.g., bimodal, trimodal, and the like) with each mode (i.e., local maxima) being between a lower limit of about 30,000 g/mol, 50,000 g/mol, 100,000 g/mol, 250,000 g/mol, or 500,000 g/mol and an upper limit of about 2,500,000 g/mol, 2,000,000 g/mol, 1,000,000 g/mol, or 500,000 g/mol, and wherein each mode of the molecular weight distribution may range from any lower limit to any upper limit and encompass any range therebetween.

In some embodiments, the HPMF polyethylene described herein of the molded polyethylene component of the present invention may be crosslinked, which may be achieved by with chemical crosslinkers, e.g., peroxides or silanes, and/or with radiation (e.g., electron beam crosslinking). Crosslinking may, in some embodiments, be useful for, inter alia, tailoring the mechanical properties of the polyethylene compositions described herein (e.g., increasing at least some mechanical properties so as to allow for molded polyethylene components of the present invention to, in some embodiments, be useful in low load applications).

In some embodiments, polyethylene compositions suitable for use in conjunction with molded polyethylene components of the present invention may comprise HPMF polyethylene described herein and optionally further comprise ingredients selected from second polymers, compatibilizers, plasticizers, APIs, imaging agents, additives, and any combination thereof.

In some embodiments, the polyethylene compositions for use in conjunction with molded polyethylene components of the present invention may comprise HPMF polyethylene described herein and optionally comprise at least one second polymer. In some instances, second polymers may be miscible, partially miscible, or non-miscible with the HPMF polyethylene.

Second polymers suitable for use in conjunction with polyethylene compositions described herein may, in some embodiments, include, but are not limited to, other polyethylenes (e.g., linear low density polyethylenes (LLDPE) and low density polyethylenes (LDPE)), polypropylenes, polybutylene, graft-modified olefin polymers, chlorinated polyethylenes, thermoplastic vulcanizates, polyether ether ketone (PEEK), polyisoprenes, polyesters, polyamides, ethylene copolymers, ethylene vinyl acetate copolymers, ethylene vinyl-methacrylate copolymers, silicones, polyethylene glycols, polyethylene oxide (PEO), ethylene oxide-propylene oxide copolymers (include block copolymers like PLURONICS® (polyethylene oxide-polypropylene oxide-polyethylene oxide triblock polymers, available from BASF)), polyethylene-polypropylene glycol (e.g., poloxamer), carbomer, polycarbophil, polyethylene imine, polyurethanes, polyacrylonitrile, styrene block copolymers, rubbers, ethylene-carboxylic acid copolymers, ethylene acrylate copolymers, polybutylene, polybutadiene, nylons, polycarbonates, ethylene ethylacrylate polymers (EEA), ethylene styrene interpolymers (ESI), aliphatic polyesters, poly(lactic acid), poly(glycolic acid), poly(lactic-co-glycolic acid), poly(butylene succinate), poly(caprolactone), polyanhydrides, poly(vinyl alcohol), cellulosics, chitans, chitosans, cellulose esters, cellulose acetate, nitrocellulose, methyl cellulose, polyvinyl pyrrolidone (PVP), hydroxyalkyl celluloses (e.g., hydroxypropyl cellulose (HPC), hydroxyethyl cellulose (HEC), hydroxymethyl cellulose, and hydroxypropyl methylcellulose (HPMC)), carboxymethyl cellulose, sodium carboxymethyl cellulose, methylcellulose, hydroxyethyl methylcellulose, hydroxypropyl methylcellulose, polyacrylates, polyacrylamides, polymethacrylamides, polyphosphazines, polyoxazolidines, polyhydroxyalkylcarboxylic acids, alginic acids (e.g., carrageenate alginates, ammonium alginate, and sodium alginate), starch and starch derivatives, polysaccharides, carboxypolymethylene, and the like, any derivative thereof, any copolymer thereof, and any combination thereof.

In some embodiments, second polymers for use in conjunction with the polyethylene compositions described herein may be absorbable. As used herein, the term “absorbable compositions” and the like refers to compositions that are capable of being absorbed by the local environment, where absorption may be of a complete composition or a component thereof (e.g., a polymer or a component thereof that may be produced if the polymer is degraded, e.g., by hydrolysis). As used herein, the term “bioabsorbable” and the like refers to absorbable compositions where the local environment is biological in nature, e.g., in vivo. The use of absorbable second polymers may, in some embodiments, allow for the formation of the pores and/or void spaces in situ. Further, the use of absorbable additional polymers may, in some embodiments, enhance the release of active pharmaceutical ingredients, described further herein. Suitable bioabsorbable second polymers for use in conjunction with the present invention may include, but are not limited to, aliphatic polyesters, poly(lactic acid), poly(glycolic acid), poly(lactic-co-glycolic acid), poly(butylene succinate), poly(caprolactone), polyanhydrides, poly(vinyl alcohol), starches, cellulosics, chitans, chitosans, cellulose esters, cellulose acetate, nitrocellulose, and the like, any derivative thereof, and any combination thereof.

In some embodiments, second polymers may be included in the polyethylene compositions described herein in an amount ranging from a lower limit of about 0.005%, 0.01%, 0.1%, 0.5%, 1%, 2%, or 5% to an upper limit of about 50%, 25%, 10%, or 5% by weight of the polyethylene composition, wherein the amount may range from any lower limit to any upper limit and encompass any subset therebetween. The amount of second polymer may depend on a number of factors that include, inter alia, the biomedical device in which the molded polyethylene component will be utilized, the desired properties (e.g., strength) of the molded polyethylene component, the composition of the second polymer, and any additional ingredients utilized in conjunction with the polyethylene composition.

In some embodiments, the polyethylene compositions for use in conjunction with molded polyethylene components of the present invention may comprise HPMF polyethylene described herein and optionally comprise at least one plasticizer and/or compatibilizer. Plasticizers and/or compatibilizers may, in some embodiments, be useful for, inter alia, tailoring the mechanical properties of the polyethylene compositions described herein and/or aiding in the homogeneous incorporation of additional ingredients into the polyethylene compositions. Examples of plasticizers and/or compatibilizers suitable for use in conjunction with polyethylene compositions described herein may include, but are not limited to, polydimethylsiloxane, polyvinylbutyral, fluoropolymers, polyvinylidene fluoride, polybutadiene, polyisoprene, polyvinyl pyrrolidone, and the like, any derivative thereof, any copolymer thereof, and any combination thereof.

In some embodiments, plasticizers and/or compatibilizers may be included in polyethylene compositions described herein in an amount ranging from a lower limit of about 0.005%, 0.01%, 0.1%, 0.5%, 1%, 2%, or 5% to an upper limit of about 50%, 25%, 10%, or 5% by weight of the polyethylene composition, wherein the amount may range from any lower limit to any upper limit and encompass any subset therebetween. The amount of plasticizers and/or compatibilizers may depend on a number of factors that include, inter alia, the biomedical device in which the molded polyethylene component will be utilized, the desired properties (e.g., strength) of the molded polyethylene component, and any additional ingredients utilized in conjunction with the polyethylene composition.

In some embodiments, the polyethylene compositions for use in conjunction with molded polyethylene components of the present invention may comprise HPMF polyethylene described herein and optionally comprise at least one active pharmaceutical ingredient (“API”). It should be noted that the term “API” encompasses prodrugs.

In some embodiments, the APIs described herein may be dispersed in at least a portion of the polyethylene compositions described herein and/or disposed on at least a portion of a surface of the polyethylene compositions described herein. By way of nonlimiting example, a biomedical device (e.g., an artificial joint) may comprise a molded polyethylene component (e.g., a base-plate stem cap) that consists essentially of a polyethylene composition that comprises a HPMF polyethylene and an active pharmaceutical, like an anti-inflammatory. Additional nonlimiting examples of APIs suitable for use in conjunction with the polyethylene composition of molded polyethylene components of the present invention are described further herein.

In some embodiments, APIs may be included in polyethylene compositions herein in an amount ranging from a lower limit of about 0.005%, 0.01%, 0.1%, 0.5%, 1%, or 2% to an upper limit of about 10%, 5%, or 2% by weight of the polyethylene composition described herein, wherein the amount may range from any lower limit to any upper limit and encompass any subset therebetween. The amount of APIs may depend on a number of factors that include, inter alia, the biomedical device in which the molded polyethylene component will be utilized and any additional ingredients utilized in conjunction with the polyethylene composition.

In some embodiments, the polyethylene compositions for use in conjunction with molded polyethylene components of the present invention may comprise HPMF polyethylene described herein and optionally comprise at least one imaging agent. As used herein, the term “imaging agent” refers to a molecule, compound, or particle that interacts with electromagnetic radiation to enable one to ascertain an image.

In some embodiments, the imaging agents described herein may be dispersed in at least a portion of the polyethylene compositions described herein and/or disposed on at least a portion of a surface of the polyethylene compositions described herein.

Examples of imaging agents suitable for use in conjunction with the polyethylene compositions described herein may, in some embodiments, include, but are not limited to, magnetic resonance imaging agents (e.g., barium sulfate, iron oxide particles, gadolinium compounds, erbium compounds, gadolinium endofullerenes, and gadolinium endonanotubes), x-ray imaging agents (e.g., barium sulfate, iodine compounds, and iodine endonanotubes), ultrasound imaging agents (e.g., perfluorocarbons and air bubbles), near infrared imaging agents (e.g., carbon nanotubes, gold nanoparticles, silver nanoparticles, and gold nanoshells), bismuth compounds, tungsten compounds, and the like, and any combination thereof.

In some embodiments, imaging agents may be useful in monitoring the condition of the molded polyethylene component of the present invention (or polyethylene composition thereof) over the long-term. By way of nonlimiting example, an imaging agent disposed on the surface of the polyethylene compositions and/or dispersed in the polyethylene composition near the surface of the molded polyethylene component may be useful for noninvasively monitoring degradation (e.g., physical wear) of the molded polyethylene component.

In some embodiments, imaging agents may be included in polyethylene compositions described herein in an amount ranging from a lower limit of about 0.005%, 0.01%, 0.1%, 0.5%, 1%, 2%, or 5% to an upper limit of about 25%, 10%, 5%, or 1% by weight of the polyethylene compositions described herein, wherein the amount may range from any lower limit to any upper limit and encompass any subset therebetween. The amount of imaging agents may depend on a number of factors that include, inter alia, the biomedical device in which the molded polyethylene component will be utilized and any additional ingredients utilized in conjunction with the polyethylene composition.

In some embodiments, the polyethylene compositions for use in conjunction with molded polyethylene components of the present invention may comprise HPMF polyethylene described herein and optionally comprise at least one additive. In some embodiments, the additives may be dispersed in at least a portion of the polyethylene compositions described herein and/or disposed on at least a portion of a surface of the polyethylene compositions described herein.

Examples of additives suitable for use in conjunction with the polyethylene compositions described herein may, in some embodiments, include, but are not limited to, antioxidants, reinforcing fillers, pigments and/or dyes, radioopaque fillers, lubricants, processing aids, light stabilizers, neutralizers, antiblocks, antifouling agents, and the like, and any combination thereof.

Antioxidants may, in some embodiments, mitigate oxidation and/or chemical degradation of polyethylene compositions described herein during storage, transportation, and/or implementation (in vivo and/or ex vivo). Examples of antioxidants suitable for use in conjunction with the polyethylene compositions described herein may, in some embodiments, include, but are not limited to, ascorbic acid, glutathione, lipoic acid, uric acid, resveratrol, flavonoids, carotenes (e.g., beta-carotene), carotenoids, tocopherols (e.g., alpha-tocopherol), tocotrienols, ubiquinol, melatonin, secondary aromatic amines, benzofuranones, hindered phenols, polyphenols, hindered amines, organophosphorus compounds, thioesters, benzoates, lactones, hydroxylamines, and any combination thereof.

Reinforcing fillers may, in some embodiments, inter alia, enhance the mechanical strength of polyethylene compositions described herein. Examples of reinforcing fillers suitable for use in conjunction with the polyethylene compositions described herein may, in some embodiments, include, but are not limited to, glass spheres, glass fibers, bioglass, graphite, aluminum powder, talc, chalk, silicates, carbonates, calcium carbonate, alumina trihydrate, marble dust, cement dust, clay, feldspar, fumed silica, alumina, magnesium oxide, magnesium hydroxide, antimony oxide, zinc oxide, barium sulfate, aluminum silicate, calcium silicate, titanium dioxide, titanates, glass microspheres, carbon fibers, metallic fibers, carbon nanotubes, wood flour, carbon black, and the like, and any combination thereof.

Examples of pigments and/or dyes suitable for use in conjunction with polyethylene compositions described herein may, in some embodiments, include, but are not limited to, inorganic-based colorants, organic-based colorants, and the like, and any combination thereof.

Examples of antifouling agents suitable for use in conjunction with polyethylene compositions described herein may, in some embodiments, include, but are not limited to, sulfoamides, penicillin, cephalosorins, carbapenems, quinolones, oxazolidones, quanternary ammonium compounds, nobel metals (e.g., silver, gold, copper, and the like), amine containing polymers, and the like, and any combination thereof.

In some embodiments, any of the additives described herein may be included in polyethylene compositions described herein in an amount ranging from a lower limit of about 0.005%, 0.01%, 0.1%, 0.5%, 1%, 2%, or 5% to an upper limit of about 50%, 25%, 10%, 5%, or 1% by weight of the polyethylene composition, wherein the amount may range from any lower limit to any upper limit and encompass any subset therebetween. The amount of additives may depend on a number of factors that include, inter alia, the biomedical device in which the molded polyethylene component will be utilized, the desired properties (e.g., strength) of the polyethylene composition, and any additional ingredients utilized in conjunction with the polyethylene composition.

In some embodiments, polyethylene compositions described herein may have a tensile modulus ranging from a lower limit of about 1 MPa, 10 MPa, 25 MPa, 50 MPa, 100 MPa, or 250 MPa to an upper limit of about 2500 MPa, 1500 MPa, 1000 MPa, 750 MPa, 500 MPa, or 250 MPa, and wherein the tensile modulus may range from any lower limit to any upper limit and encompass any subset therebetween.

In some embodiments, polyethylene compositions described herein may have a Young's modulus (elastic modulus) ranging from a lower limit of about 200 MPa, 500 MPa, 1000 MPa, or 1500 MPa to an upper limit of about 6000 MPa, 5000 MPa, 2500 MPa, or 1000 MPa, and wherein the Young's modulus may range from any lower limit to any upper limit and encompass any subset therebetween.

In some embodiments, polyethylene compositions suitable for use in conjunction with molded polyethylene components of the present invention may comprise HPMF polyethylene (according to any combination of a melt flow index described herein, an ash content described herein, and a specific gravity described herein) (which may, in some embodiments, be compliant with USP Class IV and/or ISO 10993 standards at the filing date of this application) and optionally further comprise ingredients selected from second polymers, compatibilizers, plasticizers, APIs, imaging agents, additives, and any combination thereof (including any combination thereof at concentrations described herein), and optionally have a tensile modulus and/or Young's modulus described herein.

In some embodiments, molded polyethylene components of the present invention may comprise polyethylene compositions described herein and optionally further comprise a reinforcing structure. In some embodiments, the polyethylene compositions may be disposed on at least a portion of a surface of a reinforcing structure. Reinforcing structures may, in some embodiments, inter alia, enhance the mechanical strength of the molded polyethylene component of the present invention, enable post-production shaping of the molded polyethylene component of the present invention, enable securing of the molded polyethylene component within a biomedical device and/or to another object (e.g., a bone), and any combination thereof.

Reinforcing structures suitable for use in conjunction with molded polyethylene components of the present invention may, in some embodiments, comprise ultra high molecular weight polyethylene, metals (e.g., titanium), metal alloys (e.g., cobalt chromium alloys and trabecular metal), ceramics, glass, bioglass, and the like, and any combination thereof. Reinforcing structures suitable for use in conjunction with the molded polyethylene components of the present invention may, in some embodiments, be in the form of a rod, a tube, wire, mesh, a sheet, beads, and the like, and any hybrid thereof.

In some embodiments, the molded polyethylene components of the present invention may comprise a surface coating. It should be noted that the term “coating” does not imply 100% surface coverage. In some embodiments, the surface coating may be a polymeric layer disposed on at least a portion of the surface of the molded polyethylene components.

Polymers suitable for use in conjunction with surface layers described herein may include, but are not limited to, a second HPME polyethylene, a second polymer described herein, and any combination thereof. In some embodiments, a surface layer may comprise a bioabsorbable polymer, e.g., those described herein.

In some embodiments, a surface layer (e.g., a polymeric layer) may be involved with at least one of: controlling the release profile of an API, providing burst release in the release profile of an API, delaying release of an API, and any combination thereof.

In some instances, the surface (or portion thereof) of the molded polyethylene component may be treated to enhance adhesion between the molded polyethylene component and the surface layer. Suitable treatments may include, but are not limited to, plasma treatment, corona treatment, and the like.

In some instances, the polyethylene composition may be adjusted to enhance adhesion between the molded polyethylene component and the surface layer, e.g., by inclusion of a second polymer.

In some embodiments, the molded polyethylene components of the present invention have a desired shape. As should be recognized by one of ordinary skill in the art, the desired shape may depend on, inter alia, the biomedical device in which the molded polyethylene components of the present invention are utilized. By way of nonlimiting example, in some embodiments, a molded polyethylene component, e.g., a sheath to a wire or a portion of a drug delivery device, may be in the shape of a conduit. By way of another nonlimiting example, in some embodiments, a molded polyethylene component (e.g., an aneurysm clamp) may be in a more complex shape that is designed for both movement and locking. By way of yet another nonlimiting example, in some embodiments, a molded polyethylene component (e.g., an ocular orbital wall implant or transdermal patch) may be in a shape similar to that of a sheet. By way of another nonlimiting example, in some embodiments, a molded polyethylene component (e.g., cranio-maxillofacial implant) may be in a shape similar to that of a bone or the like.

In some embodiments, a molded polyethylene component of the present invention may be nonload-bearing.

In some embodiments, molded polyethylene components of the present invention may be utilized in assembling biomedical devices. In some embodiments, a biomedical device comprising at least one molded polyethylene component of the present invention may be a load-bearing or nonload-bearing biomedical device. As used herein, the term “load-bearing” refers to a component, device, or material that is capable of maintaining a structural load. For example, in biomedical devices, a hip implant, a tibia implant, a wrist or hand implant, and the like are considered load-bearing biomedical devices.

Examples of biomedical devices that may comprise molded polyethylene components of the present invention may, in some embodiments, include, but are not limited to, chips, RFID tags, tubing, pumps, feeding tubes, catheters, vascular catheters, prosthesis, inflatable balloons, stents, heart valves, neurostimulators, cochlear implants, cranio-maxillofacial implants, synthetic cartilage, stomach rings, surgical instruments, blood vessel clamps, aneurysm clamps, spinal plugs for use in conjunction with a joint fusion system, base plates for use in artificial joints, muscle implants, nasopharyngeal implants, laryngeal implants, drug delivery devices, transdermal patches, subdermal implants, oromucosal inserts, intrauterine devices, intravaginal rings, dental fibers, and the like.

In some embodiments, molded polyethylene components of the present invention may be a conduit that comprises at least one wall defining an internal volume, the at least one wall comprising polyethylene compositions described herein.

In some embodiments, a conduit molded polyethylene component may be designed to allow fluid to flow through the internal volume. For example, biomedical devices like pumps, tubings, feeding tubes, catheters, vascular catheters, stents, heart valves, surgical instruments (e.g., surgical suction instruments), nasopharyngeal implants, and laryngeal implants may each comprise at least one conduit molded polyethylene component designed to allow fluid to flow through the internal volume.

In some embodiments, a conduit molded polyethylene component may have at least one wire disposed within the internal volume. By way of nonlimiting example, in some embodiments, a biomedical device (e.g., a pacemaker) may comprise at least one wire having a conduit molded polyethylene component disposed thereabout.

In some embodiments, molded polyethylene components described herein may be the primary composition of a biomedical device (e.g., cranio-maxillofacial implants). In some instances, the polyethylene compositions of the molded polyethylene components may comprise HPMF polyethylene and a bioabsorbable polymer, such that when implemented, the bioabsorbable polymer is absorbed leaving voids in the polyethylene composition.

In some embodiments, molded polyethylene components of the present invention may substantially encase other components of a biomedical device. In some embodiments, the encasement may be such that when implanted in a patient the surrounding tissue may be primarily exposed to the molded polyethylene component. In some embodiments, encasement may be achieved by over molding, thermoforming, or shrink wrapping a polyethylene composition described herein at least partially about the other components to be encased. In some embodiments, encasement may be achieved by forming a molded polyethylene component appropriately sized for placement of a biomedical device and/or component thereof therein.

By way of nonlimiting example, in some embodiments, a biomedical device, e.g., a cochlear implant or an RFID tag, may be substantially encased in a molded polyethylene component, such that the molded polyethylene component resembles a coating disposed substantially about the biomedical device. By way of another nonlimiting example, a biomedical device, e.g., a pacemaker or a neurostimulator, may comprise a molded polyethylene component that comprises a wire having a coating disposed substantially thereabout, the coating comprising a polyethylene composition described herein. By way of another nonlimiting example, the power supply for a neurostimulator may comprise a molded polyethylene component substantially encasing the power supply, e.g., achieved by over molding, thermoforming, or shrink wrapping.

In some embodiments, molded polyethylene components described herein may be a drug delivery device or portion thereof. In some embodiments, a transdermal patch may comprise a backing, a medicated layer, and a release layer (which may optionally comprise APIs), wherein the molded polyethylene component may be the medicated layer and/or the release layer. By way of nonlimiting example, the medicated layer may be a molded polyethylene component (e.g., comprising HPMF polyethylene and an API) and the release layer may comprise a second polymer (e.g., an ethylene copolymer like ethylene vinyl acetate). In some instances, the molded polyethylene component may comprise HPMF polyethylene and a second polymer. In some instances, the medicated layer and the release layer may independently be molded polyethylene components with different compositions (e.g., different HPMF polyethylene, optionally blended with a second polymer, optionally comprising an API or another additive, and so on).

Similar to the transdermal patch examples, other drug delivery devices may comprise molded polyethylene components. For example, a vaginal ring with a core similar to the medicated layer above and the sheath similar to the release layer above. In another example, a subdermal implant like a rod or sheet with a multi-layer structure may have at least one layer similar to the medicated layer and at least one layer similar to the release layer described above.

Forming molded polyethylene components of the present invention into a desired shape may involve extruding, injection molding, blow molding, over molding, thermoforming, shrink wrapping, compression molding, casting, calendaring, near net shape molding, adhesive bonding, mechanical fastening, and the like, any hybrid thereof, and any combination thereof.

In some embodiments, forming molded polyethylene components of the present invention may involve extruding (or the like as described above) a polyethylene composition in melt form (i.e., a polyethylene composition melt) into a desired shape. In some embodiments, a polyethylene composition melt suitable for use in forming a molded polyethylene component may comprise HPMF polyethylene described herein and any suitable optional ingredients (e.g., second polymers, plasticizers, compatibilizers, APIs, imaging agents, additives, and the like).

Temperatures suitable for extruding (or the like as described above) a polyethylene composition melt described herein may, in some embodiments, range from a lower limit of about 60° C., 75° C., or 100° C. to an upper limit of about 300° C., 250° C., 200° C., or 100° C., and wherein the temperature may range from any lower limit to any upper limit and encompass any subset therebetween. It should be noted that forming methods that utilize pressure, like compression molding, may, in some embodiments, enable forming molded polyethylene components of the present invention at lower temperatures and/or shorter times.

In some embodiments, forming molded polyethylene components of the present invention may involve extruding (or the like as described above) a polyethylene composition melt onto at least a portion of a surface of a reinforcing structure described herein.

In some embodiments, forming molded polyethylene components of the present invention may involve extruding (or the like as described above) a polyethylene composition melt onto and/or at least partially about (e.g., to encase) other components of a biomedical device (e.g., encasing conductive wires, applying a surface layer to at least one side of a metal plate, or substantially over molding a pacemaker).

In some embodiments, forming molded polyethylene components of the present invention may involve extruding (or the like as described above) a polyethylene composition melt according to any embodiments described herein into a desired shape and then treating the desired shape with suitable optional ingredients (e.g., APIs, imaging agents, additives like antioxidants, dyes, and pigments, and the like). Treating the desired shape after forming by polymer melt methods may, in some embodiments, be advantageously utilized when the suitable optional ingredients are temperature sensitive (e.g., thermally degrade). Further, treating after forming by polymer composition melt methods may advantageously allow for placement of the suitable optional ingredients on at least a portion of the surface of the molded polyethylene component and/or dispersed in and near the surface of the molded polyethylene component.

In some embodiments, forming molded polyethylene components of the present invention may involve thermoforming (or the like as described above) a sheet comprising a polyethylene composition on or about at least a portion of a biomedical device or component thereof. In some embodiments, thermoforming may involve compressing and heating a sheet (e.g., a molded polyethylene composition in sheet form) between at least two surfaces so as to form a biomedical device or component thereof. In some embodiments, at least one of the surfaces may be at least a portion of a surface of a biomedical device or component thereof. Some embodiments may involve stretching and/or orienting a sheet in the longitudinal and/or transverse directions on or about a biomedical device or component thereof, the sheet comprising a polyethylene composition; and heating the sheet.

In some embodiments, the shape of biomedical devices may be manipulated after forming, e.g., minor changes to curvature to cranio-maxillofacial implants to match a desired bone structure. Shaping biomedical devices may be performed by applying heat and pressure to the biomedical device or component thereof.

In some instances forming molded polyethylene components may involve foaming the polyethylene composition. Foaming polyethylene compositions may be by any suitable method, e.g., with pore forming agents or fluid introduction during extrusion.

Suitable pore forming compounds may include, but are not limited to, the bioabsorbable polymers described herein. Some embodiments may involve forming a molded polyethylene component from a polyethylene composition comprising a HPMF polyethylene and a bioabsorbable polymer; extracting a portion of the bioabsorbable polymer from the molded polyethylene component (e.g., via exposure to an aqueous fluid optionally with a desired pH, temperature, and salinity for extraction), thereby yielding a foamed polyethylene component.

Some embodiments may involve extruding a polyethylene component from a polyethylene composition melt; and introducing a pore forming fluid into the polyethylene composition melt while extruding. Pore forming fluids suitable for foaming polyethylene compositions described herein may include, but are not limited to, air, an inert gas (e.g., helium, nitrogen, argon, carbon dioxide, n-butane, or isobutane), volatile liquids (e.g., water, methanol, or acetone), hydrocarbons (e.g., butane, isobutane, or pentane), halogenated hydrocarbons, perfluorocarbons, and the like, or any mixture thereof. In some embodiments, the pore forming fluids may be in a gas, liquid, subcritical, or supercritical form dissolved in the polyethylene composition melt. In some embodiments, pore forming fluids may serve to form the pores and as an agent, e.g., a perfluorocarbon gas that provides contrast in ultrasound imaging. In some embodiments, pore forming fluids may be a volatile liquid that serves to form the pores and plasticize the polyethylene composition melt. In some embodiments, the amount of pore forming fluids added to a polyethylene composition melt may be at or below the saturation point of the pore forming fluids in the polyethylene composition melt.

The parameters of introducing pore forming fluids into the polyethylene composition melt may be controlled to provide control over the diameter distribution of the pores of the resultant molded polyethylene component. Suitable parameters to adjust may include, but are not limited to, temperature of the polyethylene composition melt, temperature of the pore forming fluids, pressure of pore forming fluids, composition of the pore forming fluids, composition of the polyethylene composition melt, pressure of the polyethylene composition melt, conditions of extrusion, and any combination thereof.

In some embodiments, biomedical devices comprising molded polyethylene components of the present invention may be implanted in and/or used in conjunction with the treatment of a patient. As used herein, the terms “subject” and “patient” are used interchangeably and refer to both human and nonhuman animals and insects. The term “nonhuman animals” as used herein includes all vertebrates, e.g., mammals and non-mammals, such as nonhuman primates, mice, rats, sheep, dogs, cats, horses, cows, chickens, amphibians, fish, reptiles, and the like. The term “insects” as used herein includes all arthropods, e.g., bees, flies, Drosophila flies, beetles, spiders, and the like.

In some embodiments, molded polyethylene components of the present invention may be included in a kit that also includes a set of instructions. By way of nonlimiting example, a conduit molded polyethylene component, e.g., a tubing or a catheter, may be included in a kit along with a set of instructions for how to properly utilize the conduit molded polyethylene component, e.g., installing the tubing into biomedical device like a pump or inserting the catheter into a patient.

In some embodiments, molded polyethylene components of the present invention may comprise polyethylene compositions that comprise HPMF polyethylene (according to any combination of a melt flow index described herein, an ash content described herein, and a specific gravity described herein) (which may, in some embodiments, be compliant with USP Class IV and/or ISO 10993 standards at the filing date of this application) and optionally further comprise ingredients selected from second polymers, compatibilizers, plasticizers, APIs, imaging agents, additives, and any combination thereof (including any combination thereof at concentrations described herein), and optionally have a tensile modulus and/or Young's modulus described herein, and the molded polyethylene components may optionally further comprise a reinforcing structure, have a desired shape, and be nonload-bearing. Such molded polyethylene components may be a biomedical device or be a component of a biomedical device. Such a biomedical device may, in some embodiments, be included in a kit and/or used for treating a patient, which may include implantation. Such molded polyethylene components may be formed according to any method or hybrid of methods described herein.

### I. High-Purity, Melt-Flowable Polyethylene

As described above, the terms “high-purity, melt-flowable polyethylene” and “HPMF polyethylene,” as used herein, refer to polyethylene having a melt flow index of about 3.5 g/10 min or greater as measured by ASTM D1238 at 190° C./21.5 kg weight and having an ash content of about 500 ppm or less.

Some embodiments of the present invention may involve producing the HPMF polyethylene described herein. In some embodiments, producing the HPMF polyethylene described herein may involve polymerizing at least one olefin (e.g., comprising ethylene and optionally at least one of propylene and/or butylene) so as to yield polyethylene having a melt flow index of about 3.5 g/10 min or greater as measured by ASTM D1238 at 190° C./21.5 kg weight, and then treating the polyethylene so as to yield HPMF polyethylene.

In some embodiments, the polymerization of at least one olefin may be carried out in suspension at low pressure and temperature in one or multiple steps in a continuous, batch, or hybrid process. In some embodiments, polymerizing the at least one olefin may optionally be performed in the presence of at least one of: molar mass regulators, catalysts (e.g., titanium catalysts, aluminum/titanium catalysts, metallocenes, post metallocenes, and alumoxane), supported catalysts, organoaluminum compounds, hydrogen, anti-fouling agents, anti-static agents, and the like, and any combination thereof.

In some embodiments, the melt flow index of the polyethylene produced may be manipulated by, inter alia, the concentration and composition of the materials/compounds present during reaction (e.g., the at least one olefin, molar mass regulators, catalysts, organoaluminum compounds, hydrogen, and the like), the polymerization temperature, the catalyst, the composition of the solvents used, and any combination thereof.

In some embodiments, the at least one olefin may be present at a partial pressure of about 10 MPa or less. In some embodiments, the at least one olefin may be present in a polymerization reaction at a partial pressure ranging from a lower limit of about 0.01 MPa, 0.05 MPa, 0.1 MPa, or 0.5 MPa to an upper limit of about 10 MPa, 7 MPa, 5 MPa, or 3 MPa, and wherein the partial pressure of the at least one olefin may range from any lower limit to any upper limit and encompass any subset therebetween.

In some embodiments, the polymerization may be performed in the presence of hydrogen such that the partial pressure ratio of the at least one olefin to hydrogen ranges from a lower limit of about 0.01:1, 0.05:1, 0.1:1, 5:1, 10:1, or 25:1 to an upper limit of about 100:1, 75:1, or 50:1, and wherein the ratio may range from any lower limit to any upper limit and encompass any subset therebetween.

In some embodiments, the polymerization may be performed at a temperature ranging from a lower limit of about 30° C., 50° C., or 70° C. to an upper limit of about 130° C., 100° C., or 90° C., and wherein the temperature may range from any lower limit to any upper limit and encompass any subset therebetween.

Solvents suitable for use in conjunction with polymerization reactions described herein may include, but are not limited to, butane, pentane, hexane, cyclohexene, octane, nonane, decane, isomers thereof, and the like, and any combination thereof.

In some embodiments, after polymerization, treating the polyethylene so as to yield the HPMF polyethylene may be achieved by treatment with at least one of: steam, an inert solvent the catalyst is at least partially soluble in (e.g., an acid, acetone, water, and a halogenated solvent), and the like, and any combination thereof, concurrently or in series. In some embodiments, the inert solvent may be chosen based on, inter alia, the molded polyethylene component to be produced with the HPMF polyethylene. For example, a halogenated solvent may be avoided or used early in a series of treatments in the production of HPMF polyethylene so as to reduce the potential for halogenated solvent contamination in the molded polyethylene components for in vivo applications.

In some embodiments, treating may further comprise elevated temperatures and/or reduced pressures to remove any reaction solvents and/or inert solvents described herein.

In some embodiments, the treating may be for a prolonged period of time so as to yield a HPMF polyethylene with an ash content of about 500 ppm or less, about 250 ppm or less, about 100 ppm or less, or most preferably about 50 ppm or less.

### II. Active Pharmaceutical Ingredients

Active pharmaceutical ingredients (“APIs”) for use in conjunction with the molded polyethylene components of the present invention may include, but are not limited to, therapeutic agents, antibiotics, antifungals, active biologicals, antitoxins, antigens, nutritional supplements, nutraceuticals, and the like, any proversion thereof, and any combination thereof.

Examples of suitable therapeutic agents for use in conjunction with the molded polyethylene components of present invention may include, but are not limited to, 16-alpha fluoroestradiol, 16-alpha-gitoxin, 16-epiestriol, 17-alpha dihydroequilenin, 17-alpha estradiol, 17-beta estradiol, 17-hydroxy progesterone, 1-alpha-hydroxyvitamin D2, 1-dodecpyrrolidinone, 20-epi-1,25 dihydroxyvitamin D3, 22-oxacalcitriol, 2CW, 2′-nor-cGMP, 3-isobutyl GABA, 5-ethynyluracil, 6-FUDCA, 7-methoxytacrine, abamectin, abanoquil, abcizimab (commercially available as REOPRO® from Eli Lilly and Company), abecarnil, abiraterone, ablukast, ablukast sodium, acadesine, acamprosate, acarbose, acebutolol, acecamide hydrochloride, aceclidine, aceclofenae, acedapsone, aceglutamide aluminum, acemannan, acenocoumarol, acetaminophen, acetazolamide, acetohexamide, acetohydroxamic acid, acetomepregenol, acetophenazine maleate, acetosulfone sodium, acetylcholine chloride, acetylcysteine, acetyl-L-carnitine, acetylmethadol, acifran, acipimox, acitemate, acitretin, acivicin, aclarubicin, aclatonium, acodazole hydrochloride, aconiazide, acrisorcin, acrivastine, acronine, actisomide, actodigin, acyclovir, acylfulvene, adafenoxate, adalimumab (commercially available as HUMIRA® from Abbott Laboratories), adapalene, adatanserin, adatanserin hydrochloride, adecypenol, adefovir, adelmidrol, ademetionine, adenosine, adinazolam, adipheinine hydrochloride, adiposin, adozelesin, adrafinil, adrenalone, airbutamine, alacepril, alamecin, alanine, alaproclate, alaptide, albendazole, albolabrin, albuterol (commercially available as VENTOLIN® from GlaxoSmithKline), albutoin, alclofenae, alclometasone dipropionate, aluminum chlorhydroxyallantoinate (commercially available as ALCOLOXA® from TRI-K Industries, Inc.), aldecalmycin, aldesleukin, aldioxa, alendronate sodium (commercially available as FOSAMAX® from Merck), alendronic acid, alentemol, alentemol hydrobromide, aletamine hydrochloride, aleuronium chloride, alexidine, alfacalcidol, alfentanil hydrochloride, alfuzosin, algestone acetonide, alglucerase, aliflurane, alinastine, alipamide, allantoin, allobarbital, allopurinol, a tachy-kinins (TK) antagonist, alonimid, alosetron, alosetron hydrochloride, alovudine, alpertine, alpha amylase, alpha idosone, alpidem, alprazolam (commercially available as XANAX® from Pfizer, Inc.), alprenolol hydrochloride, alprenoxime hydrochloride, alprostadil, alrestatin sodium, altanserin tartrate, alteplase, althiazide, altretamine, altromycin B, alverinc citrate, alvircept sudotox, amadinone acetate, amantadine hydrochloride, ambamustine, ambomycin, ambruticin, ambuphylline, ambuside, amcinafal, amcinonide, amdinocillin, amdinocillin pivoxil, amedalin hydrochloride, amelometasone, ameltolide, amesergide, ametantrone acetate, amezinium metilsulfate, amfebutamone, amfenac sodium, amflutizole, amicycline, amidephrine mesylate, amidox, amifloxacin, amifostine, amikacin, amiloride hydrochloride, aminacrine hydrochloride, aminobenzoate potassium, aminobenzoate sodium, aminocaproic acid, aminoglutethimide, aminohippurate sodium, aminolevulinic acid, aminophylline, aminorex, aminosalicylate sodium, aminosalicylic acid, amiodarone, amiprilose hydrochloride, amiquinsin hydrochloride, amisulpride, amitraz, amitriptyline hydrochloride, amlexanox, amlodipine, amobarbital sodium, amodiaquine, amodiaquine hydrochloride, amorolfine, amoxapine, amoxicillin, amphecloral, amphetamine sulfate, amphomycin, amphotericin B, ampicillin, ampiroxicam, ampyzine sulfate, amquinate, amrinone, aminone, amrubicin, amsacrine, amythiamicin, anagestone acetate, anagrelide, anakinra, ananain, anaritide, anaritide acetate, anastrozole (commercially available as ARIMIDEX® from AstraZeneca), anazolene sodium, ancrod, andrographolide, androstenedione, angiogenesis inhibitors, angiotensin amide, anidoxime, anileridine, anilopam hydrochloride, aniracetam, anirolac, anisotropine methylbromide, anistreplase, anitrazafen, anordrin, antagonist D, antagonist G, antarelix, antazoline phosphate, anthelmycin, anthralin, anthramycin, antiandrogen, antihemophilic factor (commercially available as XYNTHA® from Pfizer, Inc.), acedapsone, felbamate, antiestrogen, antineoplaston, antipyrine, antisense oligonucleotides, apadoline, apafant, apalcillin sodium, apaxifylline, apazone, aphidicolin glycinate, apixifylline, apomorphine hydrochloride, apraclonidine, apraclonidine hydrochloride, apramycin, aprindine, aprindine hydrochloride, aprosulate sodium, aprotinin, aptazapine maleate, aptiganel, apurinic acid, apurinic acid, aranidipine, aranotin, arbaprostil, arbekicin, 1-methyl-2-((phenylthio) methyl)-3-carbethoxy-4-((dimethylamino) methyl)-5-hydroxy-6-bromindole (commercially available as ARBIDOL® from Masterlek), arbutamine hydrochloride, arclofenin, ardeparin sodium, (2R,4R)-1-[(2S)-5-(diaminomethylideneamino)-2-[[(3R)-3-methyl-1,2,3,4-tetrahydroquinolin-8-yl] sulfonylamino] pentanoyl]-4-methyl-piperidine-2-carboxylic acid (commercially available as ARGATROBAN® from GlaxoSmithKline), arginine, argipressin tannate, arildone, aripiprazol, arotinolol, arpinocid, arteflene, artilide fumarate, asimadoline, aspalatone, asparaginase, aspartic acid, aspartocin, asperfuran, aspirin, aspoxicillin, asprelin, astemizole, astromicin sulfate, asulacrine, atamestane, atenolol, atevirdine, atipamezole, atiprosin maleate, atolide, atorvastatin (commercially available as LIPITOR® from Pfizer, Inc.), atosiban, atovaquone, atpenin B, atracurium besylate, atrimustine, atrinositol, atromentin, atropine, auranofin, aureobasidin A, aurothioglucose, avilamycin, avoparcin, avridine, nizatidine (commercially available as AXID® from GlaxoSmithKline), axinastatin 1, axinastatin 2, axinastatin 3, azabon, azacitidinie, azaclorzine hydrochloride, azaconazole, azadirachtine, azalanstat dihydrochloride, azaloxan fumarate, azanator maleate, azanidazole, azaperone, azaribine, azaserine, azasetron, azatadine maleate, azathioprine, azathioprine sodium, azatoxin, azatyrosine, azelaic acid, azelastine, azelnidipine, azepindole, azetepa, azimilide, azithromycin, azlocillin, azolimine, azosemide, azotomycin, aztreonam, azumolene sodium, bacampicillin hydrochloride, baccatin III, bacitracin, baclofen, bacoside A, bacoside B, bactobolamine, balanol, balazipone, balhimycin, balofloxacin, balsalazide, bambermycins, bambuterol, bamethan sulfate, bamifylline hydrochloride, bamidazole, baohuoside 1, barmastine, barnidipine, basifungin, batanopride hydrochloride, batebulast, batelapine maleate, batimastat, beauvericin, becanthone hydrochloride, becaplermin, becliconazole, beclomethasone dipropionate, befloxatone, beinserazide, belfosdil, belladonna, beloxamide, bemesetron, bemitradine, bemoradan, benapryzine hydrochloride, benazepril hydrochloride, benazeprilat, bendacalol mesylate, bendazac, bendroflumethiazide, benflumetol, benidipine, benorterone, benoxaprofen, benoxaprofen, benoxinate hydrochloride, benperidol, bentazepam, bentiromide, benurestat, benzbromarone, benzethonium chloride, benzetimide hydrochloride, benzilonium bromide, benzindopyrine hydrochloride, benzisoxazole, benzocaine, benzochlorins, benzoctamine hydrochloride, benzodepa, benzoidazoxan, benzonatate, benzoyl peroxide, benzoylpas calcium, benzoylstaurosporine, benzquinamide, benzthiazide, benztropine, benztropine mesylate, benzydamine hydrochloride, benzylpenicilloyl polylysine, bepridil, bepridil hydrochloride, beractant, beraprost, berefrine, berlafenone, bertosamil, berythromycin, besipirdine, beta-alethine, betaclamycin B, betamethasone, betamipron, betaxolol, betaxolol hydrochloride, bethanechol chloride, bethanidine sulfate, betulinic acid, bevacizumab (commercially available as AVASTIN® available from Genenetech), bevantolol, bevantolol hydrochloride, bezafibrate, bFGF inhibitor, bialamicol hydrochloride, biapenem, bicalutamide, bicifadine hydrochloride, biclodil hydrochloride, bidisomide, bifemelane, bifonazole, bimakalim, bimithil, bindarit, biniramycin, binospirone, bioxalomycin alpha2, bipenamol hydrochloride, biperiden, biphenamine hydrochloride, biriperone, bisantrene, bisaramil, bisaziridinylspermine, bis-benzimidazole A, bis-benzimidazole B, bisnafide, bisobrin lactate, bisoprolol, bispyrithione magsulfex, bistramide D, bistramide K, bistratene A, bithionolate sodium, bitolterol besylate, bivalirudin, bizelesin, bleomycin sulfate, bolandiol dipropionate, bolasterone, boldenone undecylenate, boldine, bolenol, bolmantalate, bopindolol, bosentan, boxidine, brefeldin, breflate, brequinar sodium, bretazenil, bretylium bosylate, brifentanil hydrochloride, brimonidine, brinolase, brocresine, brocrinat, brofoxine, bromadoline maleate, bromazepam, bromchlorenone, bromelains, bromfenac, brominidione, bromocriptine, bromodiphenhydramine hydrochloride, bromoxamide, bromperidol, bromperidol decanoate, brompheniramine baleate, broperamole, bropirimine, brotizolam, bucamide maleate, bucindolol, buclizine hydrochloride, bucromarone, budesonide (commercially available as RHINOCORT® and ENTOCORT® from AstraZeneca), budipine, budotitane, buformin, bumetamide, bunaprolast, bunazosin, bunolol hydrochloride, bupicomide, bupivacaine hydrochloride, buprenorphine hydrochloride, bupropion hydrochloride, buramate, buserelin acetate, buspirone hydrochloride, busulfan, butabarbital, butacetin, butaclamol hydrochloride, butalbital, butamben, butamirate citrate, butaperazine, butaprost, butedronate tetrasodium, butenafine, buterizine, buthionine sulfoximine, butikacin, butilfenin, butirosin sulfate, butixirate, butixocort propionate, butoconazole nitrate, butonate, butopamine, butoprozine hydrochloride, butorphanol, butoxamine hydrochloride, butriptyline hydrochloride, cactinomycin, cadexomer iodine, caffeine, calanolide A, calcifediol, calcipotriene, calcipotriol, calcitonin, calcitriol, calcium undecylenate, calphostin C, calusterone, cambendazole, camonagrel, camptothecin derivatives, canarypox IL-2, candesartan, candicidin, candoxatril, candoxatrilat, caniglibose, canrenoate potassium, canrenone, capecitabine, capobenate sodium, capobenic acid, capreomycin sulfate, capromab, capsaicin, captopril, capuride, caracemide, carbachol, carbadox, carbamazepine, carbamide peroxide, carbantel lauryl sulfate, carbaspirin calcium, carbazeran, carbazomycin C, carbenicillin potassium, carbenoxolone sodium, carbetimer, carbetocin, carbidopa, carbidopa-levodopa, carbinoxamine maleate, carbiphene hydrochloride, carbocloral, carbocysteine, carbol-fuchsin, carboplatin, carboprost, carbovir, carboxamide-amino-triazole, carboxyamidotriazole, carboxymethylated beta-1,3-glucan, carbuterol hydrochloride, CaRest M3, carfentanil citrate, carisoprodol, carmantadine, carmustine, CARN 700, camidazole, caroxazone, carperitide, carphenazine maleate, carprofen, carsatrin succinate, cartazolate, carteolol, carteolol hydrochloride, cartilage derived inhibitor, carubicin hydrochloride, carumonam sodium, carvedilol, carvotroline, carvotroline hydrochloride, carzelesin, casein kinase inhibitors (ICOS), castanospermine, caurumonam, cebaracetam, cecropin B, cedefingol, cefaclor, cefadroxil, cefamandole, cefaparole, cefatrizine, cefazaflur sodium, cefazolin, cefbuperazone, cefcapene pivoxil, cefdaloxime pentexil tosilate, cefdinir, cefditoren pivoxil, cefepime, cefetamet, cefetecol, cefixime, cefluprenam, cefinenoxime hydrochloride, cefinetazole, cefminlox, cefodizime, cefonicid sodium, cefoperazone sodium, ceforamide, cefoselis, cefotaxime sodium, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftibuten, ceftizoxime sodium, ceftriaxone, cefuroxime, celastrol, celikalim, celiprolol, cepacidiine A, cephacetrile sodium, cephalexin, cephaloglycin, cephaloridine, cephalothin sodium, cephapirin sodium, cephradine, cericlamine, cerivastatin, ceronapril, certoparin sodium, ceruletide, cetaben sodium, cetalkonium chloride, cetamolol hydrochloride, cetiedil, cetirizine, cetophenicol, cetraxate hydrochloride, cetrorelix, cetuximab (commercially available as ERBITUX® from Eli Lilly and Company), cetylpyridinium chloride, chenodiol, chlophedianol hydrochloride, chloral betaine, chlorambucil, chloramphenicol, chlordantoin, chlordiazepoxide, chlorhexidine gluconate, chlorins, chlormadinone acetate, chloroorienticin A, chloroprocaine hydrochloride, chloropropamide, chloroquine, chloroquinoxaline sulfonamide, chlorothiazide, chlorotrianisene, chloroxine, chloroxylenol, chlorphenesin carbamate, chlorpheniramine maleate, chlorpromazine, chlorpropamide, chlorprothixene, chlortetracycline bisulfate, chlorthalidone, chlorzoxazone, cholestyramine resin, chromonar hydrochloride, cibenzoline, cicaprost, ciclafrine hydrochloride, ciclazindol, ciclesonide, cicletanine, ciclopirox, cicloprofen, cicloprolol, cidofovir, cidoxepin hydrochloride, cifenline, ciglitazone, ciladopa hydrochloride, cilansetron, cilastatin sodium, cilazapril, cilnidipine, cilobamine mesylate, cilobradine, cilofungin, cilostazol, cimaterol, cimetidine, cimetropium bromide, cinalukast, cinanserin hydrochloride, cinepazet maleate, cinflumide, cingestol, cinitapride, cinnamedrine, cinnarizine, cinolazepam, cinoxacin, cinperene, cinromide, cintazone, cintriamide, cioteronel, cipamfylline, ciprefadol succinate, ciprocinonide, ciprofibrate, ciprofloxacin, ciprostene, ciramadol, cirolemycin, cisapride, cisatracurium besilate, cisconazole, cisplatin, cisporphyrin, cistinexine, citalopram, citenamide, citicoline, citreamicin alpha, cladribine, clamoxyquin hydrochloride, clarithromycin, clausenamide, clavulanate potassium, clazolam, clazolimine, clebopride, clemastine, Clentiazem maleate, clidinium bromide, clinafloxacin, clindamycin, clioquinol, clioxamide, cliprofen, clobazam, clobetasol propionate, clobetasone butyrate, clocortolone acetate, clodanolene, clodazon hydrochloride, clodronic acid, clof azimine, clofibrate, clofilium phosphate, clogestone acetate, clomacran phosphate, clomegestone acetate, clometherone, clomethiazole, clomifene analogues, clominorex, clomiphene, clomipramine hydrochloride, clonazepam, clonidine, clonitrate, clonixeril, clonixin, clopamide, clopenthixol, cloperidone hydrochloride, clopidogrel (commercially available as PLAVIX® from Bristol-Myers Squibb and Sanofi Pharmaceuticals), clopimozide, clopipazan mesylate, clopirac, cloprednol, cloprostenol sodium, clorazepate dipotassium, clorethate, clorexolone, cloroperone hydrochloride, clorprenaline hydrochloride, clorsulon, clortermine hydrochloride, closantel, closiramine aceturate, clothiapine, clothixamide maleate cloticasone propionate, clotrimazole, cloxacillin benzathine, cloxyquin, clozapine, cocaine, coccidioidin, codeine, codoxime, colchicine, colestimide, colestipol hydrochloride, colestolone, colforsin, colfosceril palmitate, colistimethate sodium, colistin sulfate, collismycin A, collismycin B, colterol mesylate, combretastatin A4, combretastatin analogue, complestatin, conagenin, conorphone hydrochloride, contignasterol, contortrostatin, cormethasone acetate, corticorelin ovine triflutate, corticotropin, cortisone acetate, cortivazol, cortodoxone, cosalane, costatolide, cosyntropin, cotinine, warfarin (commercially available as COUMADIN® from Bristol-Myers Squibb), coumermycin, coumarins, crambescidin 816, crilvastatin, crisnatol, cromitrile sodium, cromolyn sodium, crotamiton, cryptophycin 8, cucumariosid, cuprimyxin, curacin A, curdlan sulfate, zinc hyaluran (commercially available as CURIOSIN® from Gedeon Richter), cyclacillin, cyclazocine, cyclazosin, cyclic HPMPC, cyclindole, cycliramine maleate, cyclizine, cyclobendazole, cyclobenzaprine, cyclobut A, cyclobut G, cyclocapron, cycloguanil pamoate, cycloheximide, cyclopentanthraquinones, cyclopenthiazide, cyclopentolate hydrochloride, cyclophenazine hydrochloride, cyclophosphamide, cycloplatam, cyclopropane, cycloserine, cyclosin, cyclosporine, cyclothialidine, cyclothiazide, cyclothiazomycin, cyheptamide, cypemycin, cypenamine hydrochloride, cyprazepam, cyproheptadine hydrochloride, cyprolidol hydrochloride, cyproterone, cyproximide, cysteamine, cysteine hydrochloride, cystine, cytarabine, cytarabine hydrochloride, cytarabine ocfosfate, cytochalasin B, cytolytic factor, cytostatin, dabigatran, dacarbazine, dacliximab, dactimicin, dactinomycin, daidzein, daledalin tosylate, dalfopristin, dalteparin sodium, daltroban, dalvastatin, danaparoid, danazol, dantrolene, daphlnodorin A, dapiprazole, dapitant, dapoxetine hydrochloride, dapsone, daptomycin, darglitazone sodium, darifenacin, darlucin A, darodipine, darsidomine, darusentan, daunorubicin hydrochloride, dazadrol maleate, dazepinil hydrochloride, dazmegrel, dazopride fumarate, dazoxiben hydrochloride, debrisoquin sulfate, decitabine, deferiprone, deflazacort, dehydrocholic acid, dehydrodidemnin B, dehydroepiandrosterone, delapril, delapril hydrochloride, delavirdine mesylate, delequamine, delfaprazine, delmadinone acetate, delmopinol, delphinidin, demecarium bromide, demeclocycline, demecycline, demoxepam, denofungin, deoxypyridinoline, 2-propylpentanoic acid (commercially available as DEPAKOTE® from Abbott), deprodone, deprostil, depsidomycin, deramciclane, dermatan sulfate, desciclovir, descinolone acetonide, desflurane, desipramine hydrochloride, desirudin, deslanoside, deslorelin, desmopressin, desogestrel, desonide, desoximetasone, desoxoamiodarone, desoxycorticosterone acetate, detajmium bitartrate, deterenol hydrochloride, detirelix acetate, devazepide, dexamethasone, dexamisole, dexbrompheniramine maleate, dexchlorpheniramine maleate, dexclamol hydrochloride, dexetimide, dexfenfluramine hydrochloride, dexifosfamide, deximafen, dexivacaine, dexketoprofen, dexloxiglumide, dexmedetomidine, dexormaplatin, dexoxadrol hydrochloride, dexpanthenol, dexpemedolac, dexpropranolol hydrochloride, dexrazoxane, dexsotalol, dextrin 2-sulphate, dextroamphetamine, dextromethorphan, dextrorphan hydrochloride, dextrothyroxine sodium, dexverapamil, dezaguanine, dezinamide, dezocine, diacetolol hydrochloride, diamocaine cyclamate, diapamide, diatrizoate meglumine, diatrizoic acid, diaveridine, diazepam, diaziquone, diazoxide, dibenzepin hydrochloride, dibenzothiophene, dibucaine, dichliorvos, dichloralphenazone, dichlorphenamide, dicirenone, diclofenac sodium, dicloxacillin, dicranin, dicumarol, dicyclomine hydrochloride, didanosine, didemnin B, didox, dienestrol, dienogest, diethylcarbamazine citrate, diethylhomospermine, diethylnorspermine, diethylpropion hydrochloride, diethylstilbestrol, difenoximide hydrochloride, difenoxin, diflorasone diacetate, difloxacin hydrochloride, difluanine hydrochloride, diflucortolone, diflumidone sodium, diflunisal, difluprednate, diftalone, digitalis, digitoxin, digoxin, dihexyverine hydrochloride, dihydrexidine, dihydro-5-azacytidine, dihydrocodeine bitartrate, dihydroergotamine mesylate, hihydroestosterone, dihydrostreptomycin sulfate, dihydrotachysterol, dihydrotaxol, phenytoin (commercially available as DILANTIN® from Parke, Davis & Company), dilevalol hydrochloride, diltiazem hydrochloride, dimefadane, dimefline hydrochloride, dimenhydrinate, dimercaprol, dimethadione, dimethindene maleate, dimethisterone, dimethyl prostaglandin A1, dimethyl sulfoxide, dimethylhomospermine, dimiracetam, dimoxamine hydrochloride, dinoprost, dinoprostone, dioxadrol hydrochloride, dioxamycin, diphenhydramine citrate, diphenidol, diphenoxylate hydrochloride, diphenyl spiromustine, dipivefin hydrochloride, dipivefrin, dipliencyprone, diprafenone, dipropylnorspermine, dipyridamole, dipyrithione, dipyrone, dirithromycin, discodermolide, disobutamide, disofenin, disopyramide, disoxaril, disulfuram, ditekiren, divalproex sodium, dizocilpine maleate, dobutamine, docarpamine, docebenone, docetaxel, doconazole, docosanol, dofetilide, dolasetron, drotrecogin alfa (commercially available as XIGRIS® from Eli Lilly and Company), duloxetine hydrochloride (commercially available as CYMBALTA® from Eli Lilly and Company), ebastine, ebiratide, ebrotidine, ebselen, ecabapide, ecabet, ecadotril, ecdisteron, echicetin, echistatin, echothiophate iodide, eclanamine maleate, eclazolast, ecomustine, econazole, ecteinascidin 722, edaravone, edatrexate, edelfosine, edifolone acetate, edobacomab, edoxudine, edrecolomab, edrophonium chloride, edroxyprogesteone acetate, efegatran, eflornithine, efonidipine, egualcen, elantrine, eleatonin, elemene, eletriptan, elgodipine, eliprodil, elsamitrucin, eltenae, elucaine, emalkalim, emedastine, emetine hydrochloride, emiglitate, emilium tosylate, emitefur, emoctakin, enadoline hydrochloride, enalapril, enalaprilat, enalkiren, enazadrem, encyprate, endralazine mesylate, endrysone, enflurane, englitazone, enilconazole, enisoprost, enlimomab, enloplatin, enofelast, enolicam sodium, enoxacin, enoxacin, enoxaparin sodium, enoxaparin sodium, enoximone, enpiroline phosphate, enprofylline, enpromate, entacapone, enterostatin, enviradene, enviroxime, ephedrine, epicillin, epimestrol, epinephrine, epinephryl borate, epipropidine, epirizole, epirubicin, epitetracycline hydrochloride, epithiazide, epoetin alfa, epoetin beta, epoprostenol, epoprostenol sodium, epoxymexrenone, epristeride, eprosartan, eptastigmine, eptifibatide, equilenin, equilin, erbulozole, erdosteine, ergoloid mesylates, ergonovine maleate, ergotamine tartrate, ersentilide, ersofermin, erythritol, erythrityl tetranitrate, erythromycin, esmolol hydrochloride, esomeprazole (commercially available as NEXIUM® from AstraZeneca), esorubicin hydrochloride, esproquin hydrochloride, estazolam, estradiol, estramustine, estramustine analogue, estrazinol hydrobromide, estriol, estrofurate, estrogen agonists, estrogen antagonists, estrogens, conjugated estrogens, esterified, estrone, estropipate, esuprone, etafedrine hydrochloride, etanidazole, etanterol, etarotene, etazolate hydrochloride, eterobarb, ethacizin, ethacrynate sodium, ethacrynic acid, ethambutol hydrochloride, ethamivan, ethanolamine oleate, ethehlorvynol, ether, ethinyl estradiol, ethiodized oil, ethionamide, ethonam nitrate, ethopropazine hydrochloride, ethosuximide, ethotoin, ethoxazene hydrochloride, ethybenztropine, ethyl chloride, ethyl dibunate, ethylestrenol, ethyndiol, ethynerone, ethynodiol diacetate, etibendazole, etidocaine, etidronate disodium, etidronic acid, etifenin, etintidine hydrochloride, etizolam, etodolac, etofenamate, etoformin hydrochloride, etomidate, etonogestrel, etoperidone hydrochloride, etoposide, etoprine, etoxadrol hydrochloride, etozolin, etrabamine, etretinate, etryptamine acetate, eucatropine hydrochloride, eugenol, euprocin hydrochloride, eveminomicin, exametazine, examorelin, exaprolol hydrochloride, exemestane, exetimibe (commercially available as ZETIA® from Merck), fadrozole, faeriefungin, famciclovir, famotidine (commercially available as PEPCID® from Merck), fampridine, fantof arone, fantridone hydrochloride, faropenem, fasidotril, fasudil, fazarabine, fedotozine, felbamate, felbinac, felodipine, felypressin, fenalamide, fenamole, fenbendazole, fenbufen, fencibutirol, fenclofenac, fenclonine, fenclorac, fendosal, fenestrel, fenethylline hydrochloride, fenfluramine hydrochloride, fengabine, fenimide, fenisorex, fenmetozole hydrochloride, fenmetramide, fenobam, fenoctimine sulfate, fenofibrate, fenoldopam, fenoprofen, fenoterol, fenpipalone, fenprinast hydrochloride, fenprostalene, fenquizone, fenretinide, fenspiride, fentanyl citrate, fentiazac, fenticlor, fenticonazole, fenyripol hydrochloride, fepradinol, ferpifosate sodium, ferristene, ferrixan, ferrous sulfate, ferumoxides, ferumoxsil, fetoxylate hydrochloride, fexofenadine, fezolamine fumarate, fiacitabine, fialuridine, fibrinogen I 125, filgrastim, filipin, finasteride (commercially available as PROPECIA® from Merck), flavodilol maleate, flavopiridol, flavoxate hydrochloride, flazalone, flecamide, flerobuterol, fleroxacin, flesinoxan, flestolol sulfate, fletazepam, flezelastine, flobufen, floctafenine, flomoxef, flordipine, florfenicol, florifenine, flosatidil, flosequinan, floxacillin, floxuridine, fluasterone, fluazacort, flubanilate hydrochloride, flubendazole, flucindole, flucloronide, fluconazole, flucytosine, fludalanine, fludarabine phosphate, fludazonium chloride, fludeoxyglucose F 18, fludorex, fludrocortisone acetate, flufenamic acid, flufenisal, flumazenil, flumecinol, flumequine, flumeridone, flumethasone, flumetramide, flumezapine, fluminorex, flumizole, flumoxonide, flunarizine, flunidazole, flunisolide, flunitrazepam, flunixin, fluocalcitriol, fluocinolone acetonide, fluocinonide, fluocortin butyl, fluocortolone, fluorescein, fluorodaunorunicin hydrochloride, fluorodopa F 18, fluoroformylone, fluoroquinolones, fluorometholone, fluorouracil, fluotracen hydrochloride, fluoxetine, fluoxymesterone, fluparoxan, fluperamide, fluperolone acetate, fluphenazine decanoate, flupirtine, fluprednisolone, fluproquazone, fluprostenol sodium, fluquazone, fluradoline hydrochloride, flurandrenolide, flurazepam hydrochloride, flurbiprofen, fluretofen, flurithromycin, fluorocitabine, fluorof amide, fluorogestone acetate, flurothyl, fluoroxene, fluspiperone, fluspirilene, fluticasone propionate (commercially available as ADVAIR® from GlaxoSmithKline), fluticasone furoate, flutrimazole, flutroline, fluvastatin, fluvastatin sodium, fluvoxamine, fluzinamide, folic acid, follicle regulatory protein, folliculostatin, fomepizole, fonazine mesylate, fondaparinux, forasartan, forfenimex, forfenirmex, formestane, formocortal, formoterol, fosarilate, fosazepam, foscarnet sodium, fosfomycin, fosfonet sodium, fosinopril, fosinoprilat, fosphenyloin, fosquidone, fostedil, fostriecin, fotemustine, fuchsin, basic, fumoxicillin, fungimycin, furaprofen, furazolidone, furazolium chloride, furegrelate sodium, furobufen, furodazole, furosemide, fusidate sodium, fusidic acid, gabapentin, gadobenate dimeglumine, gadobenic acid, gadobutrol, gadodiamide, gadolinium texaphyrin, gadopentetate dimegiumine, gadoteric acid, gadoteridol, gadoversetamide, galantamine, galdansetron, galdansetron hydrochloride, gallamine triethiodide, gallium nitrate, gallopamil, galocitabine, gamfexine, gamolenic acid, ganciclovir, ganirelix, ganirelix acetate, gelatinase inhibitors, gemcadiol, gemcitabine (commercially available as GEMZAR® from Eli Lilly and Company), gemeprost, gemfibrozil, gentamicin sulfate, gentian violet, gepirone, gestaclone, gestodene, gestonorone caproate, gestrinone, gevotroline hydrochloride, girisopam, glaspimod, glaucocalyxin A, glemanserin, gliamilide, glibornuride, glicetanile sodium, gliflumide, glimepiride, glipizide, gloximonam, glucagon, glutapyrone, glutathione inhibitors, glutethimide, glyburide, glycopine, glycopril, glycopyrrolate, glyhexamide, glymidine sodium, glyoctamide, glyparamide, colloidal gold Au 198, gonadoctrinins, gonadorelin, gonadotropins, goserelin, gramicidin, granisetron, grepafloxacin, griseofulvin, guaiapate, guaithylline, guanabenz, guanabenz acetate, guanadrel sulfate, guancydine, guanethidine monosulfate, guanfacine hydrochloride, guanisoquin sulfate, guanoclor sulfate, guanoctine hydrochloride, guanoxabenz, guanoxan sulfate, guanoxyfen sulfate, gusperimus trihydrochloride, halazepam, halcinonide, halichondrin B, halobetasol propionate, halof antrine, halof antrine hydrochloride, halofenate, halofuginone hydrobromide, halomon, galopemide, galoperidol, halopredone, haloprogesterone, haloprogin, halothane, halquinols, hamycin, han menopausal gonadotropins, hatomamicin, hatomarubigin A, hatomarubigin B, hatomarubigin C, hatomarubigin D, heparin sodium, hepsulfam, heregulin, hetacillin, heteronium bromide, hexachlorophene:hydrogen peroxide, hexafluorenium bromide, hexamethylene bisacetamide, hexedine, hexobendine, hexoprenaline sulfate, hexylresorcinol, hirudin, histamine phosphate, histidine, histoplasmin, histrelin, homatropine hydrobromide, hoquizil hydrochloride, human chorionic gonadotropin, hycanthone, hydralazine hydrochloride, hydralazine polistirex, hydrochlorothiazide, hydrocodone bitartrate, hydrocortisone, hydroflumethiazide, hydromorphone hydrochloride, hydroxyamphetamine hydrobromide, hydroxychloroquine sulfate, hydroxyphenamate, hydroxyprogesterone caproate, hydroxyurca, hydroxyzine hydrochloride, hymecromone, hyoscyamine, hypericin, ibafloxacin, ibandronic acid, ibogaine, ibopamine, ibudilast, ibufenac, ibuprofen, ibutilide fumarate, icatibant acetate, ichthammol, icotidine, idarubicin, idoxifene, idoxuridine, idramantone, idraparinux, iemefloxacin, iesopitron, ifetroban, ifosfamide, ilepeimide, illimaquinone, ilmofosine, ilomastat, ilonidap, iloperidone, iloprost, imafen hydrochloride, imazodan hydrochloride, imidapril, imidazenil, imidazoacridones, imidecyl iodine, imidocarb hydrochloride, imidoline hydrochloride, imidurea, imiloxan hydrochloride, imipenem, imipramine hydrochloride, imiquimod, immunostimulant peptides, impromidine hydrochloride, indacrinone, indapamide, indecamide hydrochloride, indeloxazine hydrochloride, indigotindisulfonate sodium, indinavir, indocyanine green, indolapril hydrochloride, indolidan, indometacin, indomethacin sodium, indoprofen, indoramin, indorenate hydrochloride, indoxole, indriline hydrochloride, infliximab (commercially available as REMICADE® from Janssen Biotech, Inc.), inocoterone, inogatran, inolimomab, inositol niacinate, insulin, insulin glargine (commercially available as LANTUS® from Sanofi-Aventis), interferons, interferon beta-1a (commercially available as AVONEX® from BIOGEN), interleukins, intrazole, intriptyline hydrochloride, iobenguane, iobenzamic acid, iobitridol, iocarmate meglumine, iocarmic acid, iocetamic acid, iodamide, iodine, iodipamide meglumine, iodixanol, iodoamiloride, iodoantipyrine I 131, iodocholesterol I 131, iododoxorubicin, iodohippurate sodium I 131, iodopyracet I 125, iodoquinol, iodoxamate meglumine, iodoxamie acid, ioglicic acid, iofetamine hydrochloride I 123, iofratol, ioglucol, ioglucomide, ioglycamic acid, iogulamide, iohexyl, iomeprol, iomethin I 125, iopamidol, iopanoic acid, iopentol, iophendylate, ioprocemic acid, iopromide, iopronic acid, iopydol, iopydone, iopyrol, iosefamic acid, ioseric acid, iosulamide meglumine, iosumetic acid, iotasul, iotetric acid, iothalamate sodium, iothalamic acid, iotriside, iotrolan, iotroxic acid, iotyrosine I 131, ioversol, ioxagiate sodium, ioxaglate meglumine, ioxaglic acid, ioxilan, ioxotrizoic acid, ipazilide, ipenoxazone, ipidacrine, ipodate calcium, ipomeanol, 4-, ipratropium bromide, ipriflavone, iprindole, iprofenin, ipronidazole, iproplatin, iproxamine hydrochloride, ipsapirone, irbesartan, irinotecan, irloxacin, iroplact, irsogladine, irtemazole, isalsteine, isamoxole, isbogrel, isepamicin, isobengazole, isobutamben, isocarboxazid, isoconazole, isoetharine, isofloxythepin, isoflupredone acetate, isoflurane, isofluorophate, isohomohalicondrin B, isoleucine, isomazole hydrochloride, isomylamine hydrochloride, isoniazid, isopropamide iodide, isopropyl alcohol, isopropyl unoprostone, isoproterenol hydrochloride, isosorbide, isosorbide mononitrate, isotiquimide, isotretinoin, isoxepac, isoxicam, isoxsuprine hydrochloride, isradipine, itameline, itasetron, itazigrel, itopride, itraconazole, ivermectin, jasplakinolide, josamycin, kahalalide F, kalafungin, kanamycin sulfate, ketamine hydrochloride, ketanserin, ketazocine, ketazolam, kethoxal, ketipramine fumarate, ketoconazole, ketoprofen, ketorfanol, ketorolac, ketotifen fumarate, kitasamycin, labetalol hydrochloride, lacidipine, lacidipine, lactitol, lactivicin, laennec, lafutidine, lamellarin-n triacetate, lamifiban, lamivudine, lamotrigine, lanoconazole, LANOXIN® (digoxin, available from GlaxoSmithKline), lanperisone, lanreotide, lansoprazole (commercially available as PREVAID® from Takeda Pharmaceuticals, Inc.), latanoprost, lateritin, laurocapram, lauryl isoquinolinium bromide, lavoltidine succinate, lazabemide, lecimibide, leinamycin, lemildipine, leminoprazole, lenercept, leniquinsin, lenograstim, lenperone, lentinan sulfate, lepirudin, leptin, leptolstatin, lercanidipine, lergotrile, lerisetron, letimide hydrochloride, letrazuril, letrozole, leucine, leucomyzin, leuprolide acetate, leuprolide, leuprorelin, levamfetamine succinate, levamisole, levdobutamine lactobionate, levcromakalim, levetiracetam, levobetaxolol, levobunolol, levobupivacaine, levocabastine, levocarnitine, levodopa, levodropropizine, levofloxacin (commercially available as LEVAQUIN® from Jessen Pharmaceuticals, Inc.), levofuraltadone, levoleucovorin calcium, levomethadyl acetate, levomethadyl acetate hydrochloride, levomoprolol, levonantradol hydrochloride, levonordefrin, levonorgestrel, levopropoxyphene napsylate, levopropylcillin potassium, levormeloxifene, levorphanol tartrate, levosimendan, levosulpiride, levothyroxine sodium, levoxadrol hydrochloride, lexipafant, lexithromycin, liarozole, libenzapril, lidamidine hydrochloride, lidocaine, lidofenin, lidoflazine, lifarizine, lifibrate, lifibrol, linarotene, lincomycin, linear polyamine analogue, linogliride, linopirdine, linotroban, linsidomine, lintitript, lintopride, liothyronine I 125, liothyronine sodium, liotrix, lirexapride, lisinopril, lissoclinamide 7, lixazinone sulfate, lobaplatin, lobenzarit sodium, lobucavir, lodelaben, lodoxamide, lofemizole hydrochloride, lofentanil oxalate, lofepramine hydrochloride, lofexidine hydrochloride, lombricine, lomefloxacin, lomerizine, lometraline hydrochloride, lometrexol, lomitapide, lomofungin, lomoxicam, lomustine, lonapalene, lonazolac, lonidamine, loperamide hydrochloride, loracarbef, lorajmine hydrochloride, loratadine, lorazepam, lorbamate, lorcamide hydrochloride, loreclezole, lorglumide, lormetazepam, lornoxicam, lornoxicam, lortalamine, lorzafone, losartan (commercially available as COZAAR® from Merck), losigamone, losoxantrone, losulazine hydrochloride, loteprednol, lovastatin, loviride, loxapine, loxoribine, lubeluzole, lucanthone hydrochloride, lufironil, lurosetron mesylate, lurtotecan, luteinizing hormone, lutetium, lutrelin acetate, luzindole, lyapolate sodium, lycetamine, lydicamycin, lydimycin, lynestrenol, lypressin, lysine, lysofylline, lysostaphin, lytic peptides, maduramicin, mafenide, magainin 2 amide, magnesium salicylate, magnesium sulfate, magnolol, maitansine, malethamer, mallotochromene, mallotojaponin, malotilate, mangafodipir, manidipine, maniwamycin A, mannitol, mannostatin A, manumycin E, manumycin F, MAPK/ERK kinase (MEK) inhibitors, mapinastine, maprotiline, marimastat, masoprocol, maspin, massetolide, matrilysin inhibitors, maytansine, mazapertine succiniate, mazindol, mebendazole, mebeverine hydrochloride, mebrofenin, mebutamate, mecamylamine hydrochloride, mechlorethamine hydrochloride, meclocycline, meclofenamate sodium, mecloqualone, meclorisone dibutyrate, medazepam hydrochloride, medorinone, medrogestone, medroxalol, medroxyprogesterone (commercially available as DEPO-PROVERA® from Pfizer, Inc.), medrysone, meelizine hydrochloride, mefenamic acid, mefenidil, mefenorex hydrochloride, mefexamide, mefloquine hydrochloride, mefruside, megalomicin potassium phosphate, megestrol acetate, meglumine, meglutol, melengestrol acetate, melitracen hydrochloride, melphalan, memotine hydrochloride, menabitan hydrochloride, menoctone, menogaril, menotropins, meobentine sulfate, mepartricin, mepenzolate bromide, meperidine hydrochloride, mephentermine sulfate, mephenyloin, mephobarbital, mepivacaine hydrochloride, meprobamate, meptazinol hydrochloride, mequidox, meralein sodium, merbarone, mercaptopurine, mercufenol chloride, mercury, meropenem, mesalamine, meseclazone, mesoridazine, mesterolone, mestranol, mesuprine hydrochloride, metalol hydrochloride, metaproterenol polistirex, metaraminol bitartrate, metaxalone, meteneprost, meterelin, metformin, methacholine chloride, methacycline, methadone hydrochloride, methadyl acetate, methalthiazide, methamphetamine hydrochloride, methaqualone, methazolamide, methdilazine, methenamine, methenolone acetate, methetoin, methicillin sodium, methimazole, methioninase, methionine, methisazone, methixene hydrochloride, methocarbamol, methohexital sodium, methopholine, methotrexate, methotrimeprazine, methoxatone, methoxyflurane, methsuximide, methyclothiazide, methyl 10 palmoxirate, methylatropine nitrate, methylbenzethonium chloride, methyldopa, methyldopate hydrochloride, methylene blue, methylergonovine maleate, methylhistamine, R-alpha, methylinosine monophosphate, methylphenidate hydrochloride, methylprednisolone, methyltestosterone, methynodiol diacelate, methysergide, methysergide maleate, metiamide, metiapine, metioprim, metipamide, metipranolol, metizoline hydrochloride, metkephamid acetate, metoclopramide, metocurine iodide, metogest, metolazone, metopimazine, metoprine, metoprolol, metoquizine, metrifonate, metrizamide, metrizoate sodium, metronidazole, meturedepa, metyrapone, metyrosine, mexiletine hydrochloride, mexrenoate potassium, mezlocillin, mfonelic acid, mianserin hydrochloride, mibefradil, mibefradil dihydrochloride, mibolerone, michellamine B, miconazole, microcolin A, midaflur, midazolam hydrochloride, midodrine, mifepristone, mifobate, miglitol, milacemide, milameline, mildronate, milenperone, milipertine, milnacipran, milrinone, miltefosine, mimbane hydrochloride, minaprine, minaxolone, minocromil, minocycline, minoxidil, mioflazine hydrochloride, miokamycin, mipragoside, mirfentanil, mirimostim, mirincamycin hydrochloride, mirisetron maleate, mirtazapine, mismatched double stranded RNA, misonidazole, misoprostol, mitindomide, mitocarcin, mitocromin, mitogillin, mitoguazone, mitolactol, mitomalcin, mitomycin, mitonafide, mitosper, mitotane, mitoxantrone, mivacurium chloride, mivazerol, mixanpril, mixidine, mizolastine, mizoribine, moclobemide, modafinil, modaline sulfate, modecamide, moexipril, mof arotene, mofegiline hydrochloride, mofezolac, molgramostim, molinazone, molindone hydrochloride, molsidomine, mometasone, monatepil maleate, monensin, monoctanoin, montelukast sodium (commercially available as SINGULAIR® available from Merck), montirelin, mopidamol, moracizine, morantel tartrate, moricizine, morniflumate, morphine, morphine sulfate, morrhuate sodium, mosapramine, mosapride, motilide, motretinide, moxalactam disodium, moxazocine, moxiraprine, moxnidazole, moxonidine, mumps skin test antigen, mustard anticancer agent, muzolimine, mycaperoxide B, mycophenolic acid, myriaporone, nabazenil, nabilone, nabitan hydrochloride, naboctate hydrochloride, nabumetone, n-acetyldinaline, nadide, nadifloxacin, nadolol, nadroparin calcium, nafadotride, nafamostat, nafarelin, nafcillin sodium, nafenopin, nafimidone hydrochloride, naflocort, nafomine malate, nafoxidine hydrochloride, nafronyl oxalate, naftifine hydrochloride, naftopidil, naglivan, nagrestip, nalbuphine hydrochloride, nalidixate sodium, nalidixic acid, nalmefene, nalmexone hydrochloride, naloxone/pentazocine, naltrexone, namoxyrate, nandrolone phenpropionate, nantradol hydrochloride, napactadine hydrochloride, napadisilate, napamezole hydrochloride, napaviin, naphazoline hydrochloride, naphterpin, naproxen, naproxol, napsagatran, naranol hydrochloride, narasin, naratriptan, nartograstim, nasaruplase, natamycin, nateplase, naxagolide hydrochloride, nebivolol, nebramycin, nedaplatin, nedocromil, nefazodone hydrochloride, neflumozide hydrochloride, nefopam hydrochloride, nelezaprine maleate, nemazoline hydrochloride, nemorubicin, neomycin palmitate, neostigmine bromide, neridronic acid, netilmicin sulfate, neutral endopeptidase, neutramycin, nevirapine, nexeridine hydrochloride, niacin, nibroxane, nicardipine hydrochloride, nicergoline, niclosamide, nicorandil, nicotinyl alcohol, nicotine (commercially available as NICOTROL® NS from Pfizer, Inc.), nifedipine, nifirmerone, nifluridide, nifuradene, nifuraldezone, nifuratel, nifuratrone, nifurdazil, nifurimide, nifurpirinol, nifurquinazol, nifurthiazole, nilutamide, nilvadipine, nimazone, nimodipine, niperotidine, niravoline, niridazole, nisamycin, nisbuterol mesylate, nisin, nisobamate, nisoldipine, nisoxetine, nisterime acetate, nitarsone, nitazoxamide, nitecapone, nitrafudam hydrochloride, nitralamine hydrochloride, nitramisole hydrochloride, nitrazepam, nitrendipine, nitrocycline, nitrodan, nitrofurantoin, nitrofurazone, nitroglycerin, nitromersol, nitromide, nitromifene citrate, nitrous oxide, nitroxide antioxidant, nitrullyn, nivazol, nivimedone sodium, nizatidine, noberastine, nocodazole, nogalamycin, nolinium bromide, nomifensine maleate, noracymethadol hydrochloride, norbolethone, norepinephrine bitartrate, norethindrone, norethynodrel, norfloxacin, norflurane, norgestimate, norgestomet, norgestrel, nortriptyline hydrochloride, noscapine, novobiocin sodium, N-substituted benzaimides, nufenoxole, nylestriol, nystatin, O6-benzylguanine, obidoxime chloride, ocaperidone, ocfentanil hydrochloride, ocinaplon, octanoic acid, octazamide, octenidine hydrochloride, octodrine, octreotide, octriptyline phosphate, ofloxacin, oformine, okicenone, olanzapine (commercially available as ZYPREXA® from Eli Lilly and Company), oligonucleotides, olopatadine, olprinone, olsalazine, olsalazine sodium, olvanil, omeprazole, onapristone, ondansetron, ontazolast, oocyte maturation inhibitor, opipramol hydrochloride, oracin, orconazole nitrate, orgotein, orlislat, ormaplatin, ormetoprim, ornidazole, orpanoxin, orphenadrine citrate, osaterone, otenzepad, oxacillin sodium, oxagrelate, oxaliplatin, oxamarin hydrochloride, oxamisole, oxamniquine, oxandrolone, oxantel pamoate, oxaprotiline hydrochloride, oxaprozin, oxarbazole, oxatomide, oxaunomycin, oxazepam, oxcarbazepine, oxendolone, oxethazaine, oxetorone fumarate, oxfendazole, oxfenicine, oxibendazole, oxiconazole, oxidopamine, oxidronic acid, oxifungin hydrochloride, oxilorphan, oximonam, oximonam sodium, oxiperomide, oxiracetam, oxiramide, oxisuran, oxmetidine hydrochloride, oxodipine, oxogestone phenpropionate, oxolinic acid, oxprenolol hydrochloride, oxtriphylline, oxybutynin chloride, oxychlorosene, oxycodone, oxymetazoline hydrochloride, oxymetholone, oxymorphone hydrochloride, oxypertine, oxyphenbutazone, oxypurinol, oxytetracycline, oxytocin, ozagrel, ozolinone, paclitaxel, palauamine, paldimycin, palinavir, paliperidone (commercially available as INVEGA® from Janssen Pharmaceuticals, Inc.), paliperidone palmitate (commercially available as INVEGA® SUSTENNA® from Janssen Pharmaceuticals, Inc.), palmitoylrhizoxin, palmoxirate sodium, pamaqueside, pamatolol sulfate, pamicogrel, pamidronate disodium, pamidronic acid, panadiplon, panamesine, panaxytriol, pancopride, pancuronium bromide, panipenem, pannorin, panomifene, pantethine, pantoprazole, papaverine hydrochloride, parabactin, parachlorophenol, paraldehyde, paramethasone acetate, paranyline hydrochloride, parapenzolate bromide, pararosaniline pamoate, parbendazole, parconazole hydrochloride, paregoric, pareptide sulfate, pargyline hydrochloride, parnaparin sodium, paromomycin sulfate, paroxetine (commercially available as PAXIL® from GlaxoSmithKlein), parthenolide, partricin, paulomycin, pazelliptine, pazinaclone, pazoxide, pazufloxacin, pefloxacin, pegaspargase, pegorgotein, pelanserin hydrochloride, peldesine, peliomycin, pelretin, pelrinone hydrochloride, pemedolac, pemerid nitrate, pemetrexed, pemirolast, pemoline, penamecillin, penbutolol sulfate, penciclovir, penfluridol, penicillin G benzathine, penicillin G potassium, penicillin G procaine, penicillin G Sodium, penicillin V, penicillin V benzathine, penicillin V hydrabamine, penicillin V potassium, pentabamate, pentaerythritol tetranitrate, pentafuside, pentamidine, pentamorphone, bentamustine, pentapiperium methylsulfate, pentazocine, pentetic acid, pentiapine maleate, pentigetide, pentisomicin, pentizidone sodium, pentobarbital, pentomone, pentopril, pentosan, pentostatin, pentoxifylline, pentrinitrol, pentrozole, peplomycin sulfate, pepstatin, perflubron, perfof amide, perfosfamide, pergolide, perhexyline maleate, perillyl alcohol, perindopril, perindoprilat, perlapine, permethrin, perospirone, perphenazine, pethidine, phenacemide, phenaridine, phenazinomycin, phenazopyridine hydrochloride, phenbutazone sodium glycerate, phencarbamide, phencyclidine hydrochloride, phendimetrazine tartrate, phenelzine sulfate, phenindione, phenmetrazine hydrochloride, phenobarbital, phenoxybenzamine hydrochloride, phenprocoumon, phenserine, phensuccinal, phensuximide, phentermine, phentermine hydrochloride, phentolamine mesilate, phentoxifylline, phenyl aminosalicylate, phenylacetate, phenylalanine, phenylalanyl ketoconazole, phenylbutazone, phenylephrine hydrochloride, phenylpropanolamine hydrochloride, phenylpropanolamine polistirex, phenyramidol hydrochloride, phenyloin, phosphatase inhibitors, physostigmine, picenadol, picibanil, picotrin diolamine, picroliv, picumeterol, pidotimod, pifamine, pilocarpine, pilsicamide, pimagedine, pimetine hydrochloride, pimilprost, pimobendan, pimozide, pinacidil, pinadoline, pindolol, pinnenol, pinocebrin, pinoxepin hydrochloride, pioglitazone (commercially available as ACTOS® from Takeda Pharmaceuticals), pipamperone, pipazethate, pipecuronium bromide, piperacetazine, piperacillin sodium, piperamide maleate, piperazine, pipobroman, piposulfan, pipotiazine palmitate, pipoxolan hydrochloride, piprozolin, piquindone hydrochloride, piquizil hydrochloride, piracetam, pirandamine hydrochloride, pirarubicin, pirazmonam sodium, pirazolac, pirbenicillin sodium, pirbuterol acetate, pirenperone, pirenzepine hydrochloride, piretanide, pirfenidone, piridicillin sodium, piridronate sodium, piriprost, piritrexim, pirlimycin hydrochloride, pirlindole, pirmagrel, pirmenol hydrochloride, pirnabine, piroctone, pirodavir, pirodomast, pirogliride tartrate, pirolate, pirolazamide, piroxantrone hydrochloride, piroxicam, piroximone, pirprofen, pirquinozol, pirsidomine, prenylamine, pitavastatin (commercially available as LIVALOA® from Eli Lilly and Company), pituitary, posterior, pivampicillin hydrochloride, pivopril, pizotyline, placetin A, platinum compounds, platinum-triamine complex, plicamycin, plomestane, pobilukast edamine, podofilox, poisonoak extract, poldine methylsulfate, poliglusam, polignate sodium, polymyxin B sulfate, polythiazide, ponalrestat, porfimer sodium, porfiromycin, potassium chloride, potassium iodide, potassium permanganate, povidone-iodine, practolol, pralidoxime chloride, pramiracetam hydrochloride, pramoxine hydrochloride, pranolium chloride, prasugrel (commercially available as EFFIENT® from Eli Lilly and Company), pravadoline maleate, pravastatin, prazepam, prazosin, prazosin hydrochloride, prednazate, prednicarbate, prednimustine, prednisolone, prednisone, prednival, pregabalin (commercially available as LYRICA® from Pfizer, Inc.), pregnenolone succiniate, prenalterol hydrochloride, pridefine hydrochloride, prifelone, prilocalne hydrochloride, prilosec, primaquine phosphate, primidolol, primidone, prinivil, prinomide tromethamine, prinoxodan, prizidilol hydrochloride, proadifen hydrochloride, probenecid, probicromil calcium, probucol, procainamide hydrochloride, procaine hydrochloride, procarbazine hydrochloride, procaterol hydrochloride, prochlorperazine, procinonide, proclonol, procyclidine hydrochloride, prodilidine hydrochloride, prodolic acid, prof adol hydrochloride, progabide, progesterone, proglumide, proinsulin human, proline, prolintane hydrochloride, promazine hydrochloride, promethazine hydrochloride, propafenone hydrochloride, propagermanium, propanidid, propantheline bromide, proparacaine hydrochloride, propatyl nitrate, propentofylline, propenzolate hydrochloride, propikacin, propiomazine, propionic acid, propionylcarnitine, propiram, propiram+paracetamol, propiverine, propofol, propoxycaine hydrochloride, propoxyphene hydrochloride, propranolol hydrochloride, propulsid, propyl bis-acridone, propylhexedrine, propyliodone, propylthiouracil, proquazone, prorenoate potassium, proroxan hydrochloride, proscillaridin, prostalene, prostratin, protamine sulfate, protegrin, protirelin, protosufloxacin, protriptyline hydrochloride, proxazole, proxazole citrate, proxicromil, proxorphan tartrate, prulifloxacin, pseudoephedrine hydrochloride, desloratadine/pseudoephedrine sulfate (commercially available as CLARINEX-D® from Merck), puromycin, purpurins, pyrabrom, pyrantel, pamoate, pyrazinamide, pyrazofurin, pyrazoloacridine, pyridostigmine bromide, pyrilamine maleate, pyrimethamine, pyrinoline, pyrithione sodium, pyrithione zinc, pyrovalerone hydrochloride, pyroxamine maleate, pyrrocaine, pyrroliphene hydrochloride, pyrroinitrin, pyrvinium pamoate, quadazocine mesylate, quazepam, quazinone, quazodine, quazolast, quetiapine (commercially available as SEROQUEL® available from AstraZenica), quiflapon, quinagolide, quinaldine blue, quinapril, quinaprilat, quinazosin hydrochloride, quinbolone, quinctolate, quindecamine acetate, quindonium bromide, quinelorane hydrochloride, quinestrol, quinfamide, quingestanol acetate, quingestrone, quinidine gluconate, quinielorane hydrochloride, quinine sulfate, quinpirole hydrochloride, quinterenol sulfate, quinuclium bromide, quinupristin, quipazine maleate, rabeprazole sodium, racephenicol, racepinephrine, raf antagonists, rafoxamide, ralitoline, raloxifene, raltitrexed, ramatroban, ramipril, ramoplanin, ramosetron, ranelic acid, ranimycin, ranitidine, ranolazine, rauwolfia serpentina, recainam, recainam hydrochloride, reclazepam, regavirumab, regramostim, relaxin, relomycin, remacemide hydrochloride, remifentanil hydrochloride, remiprostol, remoxipride, repirinast, repromicin, reproterol hydrochloride, reserpine, resinferatoxin, resorcinol, retelliptine demethylated, reticulon, reviparin sodium, revizinone, rhenium re 186 etidronate, rhizoxin, ribaminol, ribavirin, riboprine, ribozymes, ricasetron, ridogrel, rifabutin, rifametane, rifamexil, rifamide, rifampin, rifapentine, rifaximin, retinamide, rilopirox, riluzole, rimantadine, rimcazole hydrochloride, rimexolone, rimiterol hydrobromide, rimoprogin, riodipine, rioprostil, ripazepam, ripisartan, risedronate sodium, risedronic acid, risocaine, risotilide hydrochloride, rispenzepine, risperdal, risperidone, ritanserin, ritipenem, ritodrine, ritolukast, ritonavir, rizatriptan benzoate, rocastine hydrochloride, rocuronium bromide, rodocaine, roflurane, rogletimide, rohitukine, rokitamycin, roletamicide, rolgamidine, rolicyprine, rolipram, rolitetracycline, rolodine, romazarit, romurtide, ronidazole, ropinirole (commercially available as REQUIP® from GlaxoSmithKline), ropitoin hydrochloride, ropivacaine, ropizine, roquinimex, rosaramicin, rosoxacin, rotoxamine, rosuvastatin (commercially available as CRESTOR® available from AstraZeneca), roxaitidine, roxarsone, roxindole, roxithromycin, rubiginone B1, ruboxyl, rufloxacin, rupatidine, rutamycin, ruzadolane, sabeluzole, safingol, safironil, saintopin, salbutamol, salcolex, salethamide maleate, salicyl alcohol, salicylamide, salicylate meglumine, salicylic acid, salmeterol, salnacediin, salsalate, sameridine, sampatrilat, sancycline, sanfetrinem, sanguinarium chloride, saperconazole, saprisartan, sapropterin, saquinavir, sarafloxacin hydrochloride, saralasin acetate, SarCNU, sarcophytol A, sargramostim, sarmoxicillin, sarpicillin, sarpogrelate, saruplase, saterinone, satigrel, satumomab pendetide, schick test control, scopafungin, scopolamine hydrobromide, scrazaipine hydrochloride, sdi 1 mimetics, secalciferol, secobarbital, seelzone, seglitide acetate, selegiline, selegiline hydrochloride, selenium sulfide, selenomethionine se 75, selfotel, sematilide, semduramicin, semotiadil, semustine, sense oligonucleotides, sepazonium chloride, seperidol hydrochloride, seprilose, seproxetine hydrochloride, seractide acetate, sergolexole maleate, serine, sermetacin, sermorelin acetate, sertaconazole, sertindole, sertraline, setiptiline, setoperone, sevirumab, sevoflurane, sezolamide, sibopirdine, sibutramine hydrochloride, signal transduction inhibitors, silandrone, sildenafil (commercially available as VIAGRA® from Pfizer Inc.), silipide, silteplase, silver nitrate, simendan, simtrazene, simvastatin (commercially available as ZOCOR® from Merck), sincalide, sinefungin, sinitrodil, sinnabidol, sipatrigine, sirolimus, sisomicin, sitogluside, sizofuran, sobuzoxane, sodium amylosulfate, sodium iodide I 123, sodium nitroprusside, sodium oxybate, sodium phenylacetate, sodium salicylate, solverol, solypertine tartrate, somalapor, somantadine hydrochloride, somatomedin B, somatomedin C, somatrem, somatropin, somenopor, somidobove, sonermin, sorbinil, sorivudine, sotalol, soterenol hydrochloride, sparfloxacin, sparfosate sodium, sparfosic acid, sparsomycin, sparteine sulfate, spectinomycin hydrochloride, spicamycin D, spiperone, spiradoline mesylate, spiramycin, spirapril hydrochloride, spiraprilat, spirogermanium hydrochloride, spiromustine, spironolactone, spiroplatin, spiroxasone, splenopentin, spongistatin 1, sprodiamide, squalamine, stallimycin hydrochloride, stannous pyrophosphate, stannous sulfur colloid, stanozolol, statolon, staurosporine, stavudine, steffimycin, stenbolone acetate, stepronin, stilbazium iodide, stilonium iodide, stipiamide, stiripentol, stobadine, streptokinase, streptomycin sulfate, streptonicozid, streptonigrin, streptozocin, stromelysin inhibitors, strontium chloride Sr 89, succibun, succimer, succinylcholine chloride, sucralfate, sucrosof ate potassium, sudoxicam, sufentanil, sufotidine, sulazepam, sulbactam pivoxil, sulconazole nitrate, sulfabenz, sulfabenzamide, sulfacetamide, sulfacytine, sulfadiazine, sulfadoxine, sulfalene, sulfamerazine, sulfameter, sulfamethazine, sulfamethizole, sulfamethoxazole, sulfamonomethoxine, sulfamoxole, sulfanilate zinc, sulfanitran, sulfasalazine, sulfasomizole, sulfazamet, sulfinalol hydrochloride, sulfinosine, sulfinpyrazone, sulfisoxazole, sulfomyxin, sulfonterol hydrochloride, sulfoxamine, sulinldac, sulmarin, sulnidazole, suloctidil, sulofenur, sulopenem, suloxifen oxalate, sulpiride, sulprostone, sultamicillin, sulthiame, sultopride, sulukast, sumarotene, sumatriptan, suncillin sodium, suproclone, suprofen, suradista, suramin, surfomer, suricamide maleate, suritozole, suronacrine maleate, suxemerid sulfate, swainsonine, symakalim, symclosene, symetine hydrochloride, synthetic glycosaminoglycans, tadalafil (commercially available as CIALIS® and ACIRCA® from from Eli Lilly and Company), taciamine hydrochloride, tacrine hydrochloride, tacrolimus, talampicillin hydrochloride, taleranol, talisomycin, tallimustine, talmetacin, talniflumate, talopram hydrochloride, talosalate, tametraline hydrochloride, tamoxifen (commercially available as NOLVADEX® from AstraZeneca), tampramine fumarate, tamsulosin hydrochloride, tandamine hydrochloride, tandospirone, tapgen, taprostene, tasosartan, tauromustine, taxane, taxoid, tazadolene succinate, tazanolast, tazarotene, tazifylline hydrochloride, tazobactam, tazofelone, tazolol hydrochloride, tebufelone, tebuquine, technetium Tc 99 m bicisate, teclozan, tecogalan sodium, teecleukin, teflurane, tegafur, tegretol, teicoplanin, telenzepine, tellurapyrylium, telmesteine, telmisartan, telomerase inhibitors, teloxantrone hydrochloride, teludipine hydrochloride, temafloxacin hydrochloride, tematropium methyl sulfate, temazepam, temelastine, temocapril, temocillin, temoporfin, temozolomide, tenofovir, tenidap, teniposide, tenosal, tenoxicam, tepirindole, tepoxalin, teprotide, terazosin, terbinafine, terbutaline sulfate (commercially available as BRICANYL® from AstraZeneca), terconazole, terfenadine, terflavoxate, terguride, teriparatide acetate, terlakiren, terlipressin, terodiline, teroxalene hydrochloride, teroxirone, tertatolol, terutroban, tesicam, tesimide, testolactone, testosterone, tetracaine, tetrachlorodecaoxide, tetracycline, tetrahydrozoline hydrochloride, tetramisole hydrochloride, tetrazolast meglumine, tetrazomine, tetrofosmin, tetroquinone, tetroxoprim, tetrydamine, thaliblastine, thalidomide, theofibrate, theophylline, thiabendazole, thiamiprine, thiamphenicol, thiamylal, thiazesim hydrochloride, thiazinamium chloride, thiazolidinedione, thiethylperazine, thimerfonate sodium, thimerosal, thiocoraline, thiofedrine, thioguanine, thiomarinol, thiopental sodium, thioperamide, thioridazine, thiotepa, thiothixene, thiphenamil hydrochloride, thiphencillin potassium, thiram, thozalinone, threonine, thrombin, thrombopoietin, thrombopoietin mimetic, thromboxane synthase inhibitors, thromboxane receptor, antagonists, thymalfasin, thymopoietin receptor agonist, thymotrinan, thyromedan hydrochloride, thyroxine 1 125, thyroxine 1 131, tiacrilast, tiacrilast sodium, tiagabine, tiamenidine, tianeptine, tiapafant, tiapamil hydrochloride, tiaramide hydrochloride, tiazofurin, tibenelast sodium, tibolone, tibric acid, ticabesone propionate, ticarbodine, ticarcillin cresyl sodium, ticagrelor, ticlopidine, ticlatone, ticlopidine, ticrynafen, tienoxolol, tifurac sodium, tigemonam dicholine, tigestol, tiletamine hydrochloride, tilidine hydrochloride, tilisolol, tilnoprofen arbamel, tilorone hydrochloride, tiludronate disodium, tiludronic acid, timefurone, timobesone acetate, timolol, tin ethyl etiopurpurin, tinabinol, tinidazole, tinzaparin sodium, tioconazole, tiodazosin, tiodonium chloride, tioperidone hydrochloride, tiopinac, tiospirone hydrochloride, tiotidine, tiotropium bromide, tioxidazole, tipentosin hydrochloride, tipredane, tiprenolol hydrochloride, tiprinast meglumine, tipropidil hydrochloride, tiqueside, tiquinamide hydrochloride, tirandalydigin, tirapazamine, tirilazad, tirofiban, tiropramide, titanocene dichloride, tixanox, tixocortol pivalate, tizanidine hydrochloride, tobramycin, tocamide, tocamphyl, tofenacin hydrochloride, tolamolol, tolazamide, tolazoline hydrochloride, tolbutamide, tolcapone, tolciclate, tolfamide, tolgabide, lamotrigine, tolimidone, tolindate, tolmetin, tolnaftate, tolpovidone 1 131, tolpyrramide, tolrestat, tomelukast, tomoxetine hydrochloride, tonazocine mesylate, topiramate, topotecan, topotecan hydrochloride, topsentin, topterone, toquizine, torasemide, toremifene, torsemide, tosifen, tosufloxacin, totipotent stem cell factor, tracazolate, trafermin, tralonide, tramadol hydrochloride, tramazoline hydrochloride, trandolapril, tranexamic acid, tranilast, transcamide, translation inhibitors, trastuzumab (commercially available as HERCEPTIN® from Genentech), traxanox, trazodone hydrochloride, trazodone-hcl, trebenzomine hydrochloride, trefentanil hydrochloride, treloxinate, trepipam maleate, trestolone acetate, tretinoin, triacetin, triacetyluridine, triafungin, triamcinolone, triampyzine sulfate, triamterene, triazolam, tribenoside, tricaprilin, tricetamide, trichlormethiazide, trichohyalin, triciribine, tricitrates, triclofenol piperazine, triclofos sodium, triclonide, trientine, trifenagrel, triflavin, triflocin, triflubazam, triflumidate, trifluoperazine hydrochloride, trifluperidol, triflupromazine, triflupromazine hydrochloride, trifluridine, trihexyphenidyl hydrochloride, trilostane, trimazosin hydrochloride, trimegestone, trimeprazine tartrate, trimethadione, trimethaphan camsylate, trimethobenzamide hydrochloride, trimethoprim, trimetozine, trimetrexate, trimipramine, trimoprostil, trimoxamine hydrochloride, triolein 1 125, triolein 1 131, trioxifene mesylate, tripamide, tripelennamine hydrochloride, triprolidine hydrochloride, triptorelin, trisulfapyrimidines, troclosene potassium, troglitazone, trolamine, troleandomycin, trombodipine, trometamol, tropanserin hydrochloride, tropicamide, tropine ester, tropisetron, trospectomycin, trovafloxacin, trovirdine, tryptophan, tuberculin, tubocurarine chloride, tubulozole hydrochloride, tucarcsol, tulobuterol, turosteride, tybamate, tylogenin, tyropanoate sodium, tyrosine, tyrothricin, tyrphostins, ubenimex, uldazepam, undecylenic acid, uracil mustard, urapidil, urea, uredepa, uridine triphosphate, urofollitropin, urokinase, ursodiol, valaciclovir, valine, valnoctamide, valproate sodium, valproic acid, valsartan (commercially available as DIOVAN® from Novartis Pharmaceuticals), vamicamide, vanadeine, vancomycin, vaminolol, vapiprost hydrochloride, vapreotide, vardenafil (commercially available as LEVITRA® from GlaxoSmithKline), variolin B, vasopressin, vecuronium bromide, velaresol, velnacrine maleate, venlafaxine, veradoline hydrochloride, veramine, verapamil hydrochloride, verdins, verilopam hydrochloride, verlukast, verofylline, veroxan, verteporfin, vesnarinone, vexibinol, vidarabine, vigabatrin, viloxazine hydrochloride, vinblastine sulfate, vinburnine citrate, vincofos, vinconate, vincristine sulfate, vindesine, vindesine sulfate, vinepidine sulfate, vinglycinate sulfate, vinleurosine sulfate, vinorelbine, vinpocetine, vintoperol, vinxaltine, vinzolidine sulfate, viprostol, virginiamycin, viridofulvin, viroxime, vitaxin, volazocine, voriconazole, vorozole, voxergolide, warfarin sodium, xamoterol, xanomeline, xanoxate sodium, xanthinol niacinate, xemilofiban, xenalipin, xenbucin, xilobam, ximoprofen, xipamide, xorphanol mesylate, xylamidine tosylate, xylazine hydrochloride, xylometazoline hydrochloride, xylose, yangambin, zabicipril, zacopride, zafirlukast, zalcitabine, zaleplon, zalospirone, zaltidine hydrochloride, zaltoprofen, zanamivir, zankiren, zanoterone, zantac, zarirlukast, zatebradine, zatosetron, zatosetron maleate, zenarestat, zenazocine mesylate, zeniplatin, zeranol, zidometacin, zidovudine, zifrosilone, zilantel, zilascorb, zileuton, zimeldine hydrochloride, zinc undecylenate, zindotrine, zinoconazole hydrochloride, zinostatin, zinterol hydrochloride, zinviroxime, ziprasidone, zobolt, zofenopril calcium, zofenoprilat, zolamine hydrochloride, zolazepam hydrochloride, zoledronie acid, zolertine hydrochloride, zolmitriptan, zolpidem, zomepirac sodium, zometapine, zoniclezole hydrochloride, zonisamide, zopiclone, zopolrestat, zorbamyciin, zorubicin hydrochloride, zotepine, zucapsaicin, JTT-501 (PNU-182716) (reglitazar), AR-H039122, MCC-555 (netoglitazone), AR-H049020 (tesaglitazar), CS-011 (CI-1037), GW-409544x, KRP-297, RG-12525, BM-15.2054, CLX-0940, CLX-0921, DRF-2189, GW-1929, GW-9820, LR-90, LY-510929, NIP-221, NIP-223, JTP-20993, LY 29311 Na, FK 614, BMS 298585, R 483, TAK 559, DRF 2725 (ragaglitazar), L-686398, L-168049, L-805645, L-054852, demethyl asteriquinone B1 (L-783281), L-363586, KRP-297, P32/98, CRE-16336, EML-1625, pharmaceutically acceptable salts thereof (e.g., Zn, Fe, Mg, K, Na, F, CI, Br, I, acetate, diacetate, nitrate, nitrite, sulfate, sulfite, phosphate, and phosphite salts), pharmaceutically acceptable forms thereof with acid associates (e.g. HCl), and any combination thereof.

Suitable antibiotics for use in conjunction with the molded polyethylene components of the present invention may include, but are not limited to, to β-lactam antibiotics (e.g., benzathine penicillin, benzylpenicillin (penicillin G), phenoxymethylpenicillin (penicillin V), procaine penicillin, methicillin, oxacillin, nafcillin, cloxacillin, dicloxacillin, flucloxacillin, temocillin, amoxicillin, ampicillin, co-amoxiclav (amoxicillin+clavulanic acid), azlocillin, carbenicillin, ticarcillin, mezlocillin, piperacillin, cephalosporin, cephalexin, cephalothin, cefazolin, cefaclor, cefuroxime, cefamandole, cefotetan, cefoxitin, ceftriaxone, cefotaxime, cefpodoxime, cefixime, ceftazidime, cefepime, cefpirome, carbapenem, imipenem (with cilastatin), meropenem, ertapenem, faropenem, doripenem, aztreonam (commercially available as AZACTAM® from Bristol-Myers Squibb), tigemonam, nocardicin A, tabtoxinine-β-lactam, clavulanic acid, tazobactam, and sulbactam); aminoglycoside antibiotics (e.g., aminoglycoside, amikacin, apramycin, arbekacin, astromicin, bekanamycin, capreomycin, dibekacin, dihydrostreptomycin, elsamitrucin, G418, gentamicin, hygromycin B, isepamicin, kanamycin, kasugamycin, micronomicin, neomycin, netilmicin, paromomycin sulfate, ribostamycin, sisomicin, streptoduocin, streptomycin, tobramycin, verdamicin; sulfonamides such as sulfamethoxazole, sulfisomidine (also known as sulfaisodimidine), sulfacetamide, sulfadoxine, dichlorphenamide (DCP), and dorzolamide); quinolone antibiotics (e.g., cinobac, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, sparfloxacin, temafloxacin, tosufloxacin, clinafloxacin, gatifloxacin, gemifloxacin, moxifloxacin, sitafloxacin, trovafloxacin, prulifloxacin, garenoxacin, and delafloxacin); oxazolidone antibiotics (e.g., linezolid, torezolid, eperezolid, posizolid, and radezolid), and any combination thereof.

Suitable antifungals for use in conjunction with the molded polyethylene components of the present invention may include, but are not limited to, polyene antifungals (e.g., natamycin, rimocidin, filipin, nystatin, amphotericin B, candicin, and hamycin; imidazole antifungals such as miconazole (commercially available as MICATIN® from WellSpring Pharmaceutical Corporation), ketoconazole (commercially available as NIZORAL® from McNeil consumer Healthcare), clotrimazole (commercially available as LOTRAMIN® and LOTRAMIN AF® available from Merck and CANESTEN® available from Bayer), econazole, omoconazole, bifonazole, butoconazole, fenticonazole, isoconazole, oxiconazole, sertaconazole (commercially available as ERTACZO® from OrthoDematologics), sulconazole, and tioconazole; triazole antifungals such as fluconazole, itraconazole, isavuconazole, ravuconazole, posaconazole, voriconazole, terconazole, and albaconazole), thiazole antifungals (e.g., abafungin), allylamine antifungals (e.g., terbinafine (commercially available as LAMISIL® from Novartis Consumer Health, Inc.), naftifine (commercially available as NAFTIN® available from Merz Pharmaceuticals), and butenafine (commercially available as LOTRAMIN ULTRA® from Merck), echinocandin antifungals (e.g., anidulafungin, caspofungin, and micafungin), polygodial, benzoic acid, ciclopirox, tolnaftate (e.g., commercially available as TINACTIN® from MDS Consumer Care, Inc.), undecylenic acid, flucytosine, 5-fluorocytosine, griseofulvin, haloprogin, and any combination thereof.

Suitable active biologicals for use in conjunction with the molded polyethylene components of the present invention may include, but are not limited to, hormones (synthetic or natural and patient derived or otherwise), DNAs (synthetic or natural and patient derived or otherwise), RNAs (synthetic or natural and patient derived or otherwise), siRNAs (synthetic or natural and patient derived or otherwise), proteins and peptides (e.g., albumin, atrial natriuretic factor, renin, superoxide dismutase, α-1-antitrypsin, lung surfactant proteins, bacitracin, bestatin, cydosporine, delta sleep-inducing peptide (DSIP), endorphins, glucagon, gramicidin, melanocyte inhibiting factors, neurotensin, oxytocin, somostatin, terprotide, serum thymide factor, thymosin, DDAVP, dermorphin, Met-enkephalin, peptidoglycan, satietin, thymopentin, fibrin degradation product, des-enkephalin-α-endorphin, gonadotropin releasing hormone, leuprolide, α-MSH, and metkephamid), enzymes, nucleotides, oligionucleotides, antibodies, monoclonal antibodies, growth factors (e.g., epidermal growth factor (EGF), fibroblast growth factors, basic fibroblast growth factor (bFGF), nerve growth factor (NGF), bone derived growth factor (BDGF), transforming growth factors, transforming growth factor-β1 (TGF-β1), and human growth hormone (hGH)), viral surface antigens (e.g., adenoviruses, epstein-barr virus, hepatitis A virus, hepatitis B virus, herpes viruses, HIV-1, HIV-2, HTLV-III, influenza viruses, Japanese encephalitis virus, measles virus, papilloma viruses, paramyxoviruses, polio virus, rabies virus, rubella virus, vaccinia (smallpox) viruses, and yellow fever virus), bacterial surface antigens (e.g., bordetella pertussis, helicobacter pylorn, clostridium tetani, corynebacterium diphtheria, escherichia coli, haemophilus influenza, klebsiella species, legionella pneumophila, mycobacterium bovis, mycobacterium leprae, mycrobacterium tuberculosis, neisseria gonorrhoeae, neisseria meningitidis, proteus species, pseudomonas aeruginosa, salmonella species, shigella species, staphylococcus aureus, streptococcus pyogenes, vibrio cholera, and yersinia pestis), parasite surface antigens (e.g., plasmodium viva-malaria, plasmodium falciparum-malaria, plasmodium ovale-malaria, plasmodium malariae-malaria, leishmania tropica-leishmaniasis, leishmania donovani, leishmaniasis, leishmania branziliensis-leishmaniasis, trypanosoma rhodescense—sleeping sickness, trypanosoma gambiense—sleeping sickness, trypanosoma cruzi—Chagas' disease, schistosoma mansoni-schistosomiasis, schistosomoma haematobium-schistomiasis, schistosoma japonicum-shichtomiasis, trichinella spiralis-trichinosis, stronglyloides duodenale—hookworm, ancyclostoma duodenale—hookworm, necator americanus—hookworm, wucheria bancrofti-filariasis, brugia malaya-filariasis, loa loa-filariasis, dipetalonema perstaris-filariasis, dracuncula medinensis-filariasis, and onchocerca volvulus-filariasis), immunogobulins (e.g., IgG, IgA, IgM, antirabies immunoglobulin, and antivaccinia immunoglobulin), and any combination thereof.

Suitable antitoxins for use in conjunction with the molded polyethylene components of the present invention may include, but are not limited to, botulinum antitoxin, diphtheria antitoxin, gas gangrene antitoxin, tetanus antitoxin, and any combination thereof.

Suitable antigens for use in conjunction with the molded polyethylene components of the present invention may include, but are not limited to, hormones and growth factors (e.g., follicle stimulating hormone, prolactin, angiogenin, epidermal growth factor, calcitonin, erythropoietin, thyrotropic releasing hormone, insulin, growth hormones, insulin-like growth factors 1 and 2, skeletal growth factor, human chorionic gonadotropin, luteinizing hormone, nerve growth factor, adrenocorticotropic hormone (ACTH), luteinizing hormone releasing hormone (LHRH), parathyroid hormone (PTH), thyrotropin releasing hormone (TRH), vasopressin, cholecystokinin, and corticotropin releasing hormone), cytokines (e.g., interferons, interleukins, colony stimulating factors, and tumor necrosis factors: fibrinolytic enzymes, such as urokinase, kidney plasminogen activator), clotting factors (e.g., Protein C, Factor VIII, Factor IX, Factor VII and Antithrombin III), and any combination thereof.

Suitable nutritional supplements for use in conjunction with the antigents present invention may include, but are not limited to, vitamins, minerals, herbs, botanicals, amino acids, steroids, and the like.

Suitable nutraceuticals for use in conjunction with the molded polyethylene components present invention may include, but are not limited to, dietary supplements, botanicals, functional foods and extracts thereof, medicinal foods and extracts thereof, vitamins, minerals, co-enzyme Q, carnitine, multi-mineral formulas, gingseng, gingko biloba, saw palmetto, other plant-based supplements, probiotics, omega-3, canola and other oils, plant stanols, natural sweeteners, mushroom extracts, chocolate, chocolate extracts, grape extracts, berry extracts, super food extracts, quillaja molina extracts, plant extracts, yucca schidigera extract, bran, alanine, beta-carotene, carotenoids, arginin, vitamin A, asparagine, vitamin B-complex, aspartate, vitamin C, leucine, isoleucine, valine, vitamin D, citrulline, vitamin E, cysteine, vitamin K, glutamine, minerals, micro-nutrients, glutamic acid, calcium, glycine, chromium, histidine, copper, lysine, iodine, methionine, iron, ornithine, magnesium, phenylalanine, potassium, proline, selenium, serine, zinc, taurine, threonine, alpha lipoic acid, tryptophan, green tea extracts, tyrosine, essential fatty acids (EFA), whey protein, flax seed oil, and any combination thereof.

A typical dosage of APIs may range from about 0.001 mg/kg to about 1000 mg/kg, preferably from about 0.01 mg/kg to about 100 mg/kg, and more preferably from about 0.10 mg/kg to about 20 mg/kg, relative to weight of the patient. In some embodiments, an API may be used alone or in combination with another API. One skilled in the art should understand the dose and/or combination of agents should be chosen so as to minimize adverse interactions.

Embodiments disclosed herein include:

A: a biomedical device that comprises a molded polyethylene component that comprises a polyethylene composition that comprises polyethylene having a melt flow index of about 3.5 g/10 min or greater as measured by ASTM D1238 at 190° C./21.5 kg weight and having an ash content of about 500 ppm or less;

B: a biomedical device that comprises a conduit that comprises at least one wall defining an internal volume, the at least one wall comprising: polyethylene having a melt flow index of about 3.5 g/10 min or greater as measured by ASTM D1238 at 190° C./21.5 kg weight and having an ash content of about 500 ppm or less;

C: a method that comprises providing a polyethylene composition melt that comprises polyethylene having a melt flow index of about 3.5 g/10 min or greater as measured by ASTM D1238 at 190° C./21.5 kg weight and having an ash content of about 500 ppm or less; and extruding the polyethylene composition melt to form a molded polyethylene component of a biomedical device;

D: a method that comprises providing a polyethylene composition melt that comprises polyethylene having a melt flow index of about 3.5 g/10 min or greater as measured by ASTM D1238 at 190° C./21.5 kg weight and having an ash content of about 500 ppm or less; and injection molding the polyethylene composition melt to form a molded polyethylene component of a biomedical device;

E: a method that comprises providing a component of a biomedical device; and substantially encasing the component in polyethylene having a melt flow index of about 3.5 g/10 min or greater as measured by ASTM D1238 at 190° C./21.5 kg weight and having an ash content of about 500 ppm or less;

F: a method that comprises providing a sheet comprising a polyethylene composition that comprises polyethylene having a melt flow index of about 3.5 g/10 min or greater as measured by ASTM D1238 at 190° C./21.5 kg weight and having an ash content of about 500 ppm or less; and thermoforming the sheet into a molded polyethylene component of a biomedical device; and

G: a method that comprises providing a sheet comprising a polyethylene composition that comprises polyethylene having a melt flow index of about 3.5 g/10 min or greater as measured by ASTM D1238 at 190° C./21.5 kg weight and having an ash content of about 500 ppm or less; and shrink wrapping the sheet onto a portion of a biomedical device or component thereof; and

H: a method that comprises polymerizing at least one olefin in the presence of a catalyst so as to form polyethylene having a melt flow index of about 3.5 g/10 min or greater as measured by ASTM D1238 at 190° C./21.5 kg weight, the at least one olefin being selected from the group consisting of ethylene, propylene, and/or butylene, and any combination thereof; and treating the polyethylene to yield a treated polyethylene having an ash content of about 500 ppm or less.

Each of embodiments A-G may independently have one or more of the following additional elements in any combination: Element 1: the polyethylene having a specific gravity of about 0.93 g/cm3 to about 0.97 g/cm³; Element 2: the polyethylene having an average molecular weight of about 30,000 g/mol to about 2,500,000 g/mol; Element 3: the polyethylene comprising a copolymer of ethylene and at least one monomer selected from the group consisting of propylene, butylene, and any combination thereof; Element 4: the polyethylene being crosslinked; Element 5: the polyethylene composition being foamed; Element 6: the molded polyethylene component is nonload-bearing; Element 7: the biomedical device being nonload-bearing or load-bearing; Element 8: the polyethylene composition further comprising an active pharmaceutical ingredient; Element 9: the polyethylene composition further comprising an imaging agent; Element 10: the polyethylene composition further comprising at least one selected from the group consisting of a second polymer, a plasticizer, a compatibilizer, an antioxidant, a reinforcing filler, a pigment, a dye, a radioopaque filler, a lubricant, a processing aid, a light stabilizer, a neutralizer, an antiblock, and any combination thereof; Element 11: the molded polyethylene component (or the like, e.g., a sheet) further comprising a reinforcing structure, wherein the polyethylene composition is disposed on at least a portion of a surface of the reinforcing structure; Element 12: the biomedical device further comprising other components, and wherein the molded polyethylene component substantially encases the other components; Element 13: the biomedical device being at least one selected from the group consisting of a chip, an RFID tag, tubing, a pump, a feeding tube, a catheter, a vascular catheter, a prosthetic, an inflatable balloon, a stent, a heart valve, a neurostimulator, a cochlear implant, a cranio-maxillofacial implant, synthetic cartilage, a stomach ring, a surgical instrument, a blood vessel clamp, an aneurysm clip, a spinal plug for use in conjunction with a joint fusion system, a base-plate stem cap for an artificial joint, a muscle implant, a nasopharyngeal implant, and a laryngeal implant, a drug delivery device, a transdermal patch, a subdermal implant, an oromucosal insert, an intrauterine device, an intravaginal ring, and a dental fiber; Element 14: the biomedical device being in a kit with a set of instructions; Element 15: implanting the biomedical device in a patient; and Element 16: treating a patient with the biomedical device. Embodiment H may have one or more of the following additional elements in any combination: Element 17: the treated polyethylene having a specific gravity of about 0.93 g/cm3 to about 0.97 g/cm³; Element 18: the treated polyethylene having an average molecular weight of about 30,000 g/mol to about 2,500,000 g/mol; Element 19: treating involves exposing the polyethylene concurrently or in series to at least one selected from the group consisting of steam, an inert solvent the catalyst is at least partially soluble in, an acid, acetone, water, a halogenated solvent, and any combination thereof; Element 20: treating involves increased temperature and/or decreased pressure; and Element 21: forming a molded polyethylene component of a biomedical device from the treated polyethylene.

By way of non-limiting example, exemplary combinations independently applicable to embodiments A-G include: at least two of Elements 1-6 in combination; Element 8 in combination with at least one of Elements 1-6; Element 9 in combination with at least one of Elements 1-6; Element 10 in combination with at least one of Elements 1-6; Element 13 in combination with at least one of Elements 1-6; Elements 8 and 13 in combination with at least one of Elements 1-6; Elements 9 and 13 in combination with at least one of Elements 1-6; Element 1 in combination with Element 4; Elements 10 and 13 in combination with at least one of Elements 1-6; Element 13 in combination with at least two of Elements 8-10 and at least one of Elements 1-6; Element 14 in combination with the foregoing any of the combinations; Element 15 in combination with the foregoing any of the combinations; Element 16 in combination with the foregoing any of the combinations; and so on.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

1. A biomedical device comprising:
a molded polyethylene component that comprises a polyethylene composition that comprises polyethylene having a melt flow index of about 3.5 g/10 min or greater as measured by ASTM D1238 at 190° C./21.5 kg weight and having an ash content of about 500 ppm or less.

2. The biomedical device of claim 1, wherein the polyethylene has a specific gravity of about 0.93 g/cm³ to about 0.97 g/cm³.

3. The biomedical device of claim 1, wherein the polyethylene has an average molecular weight of about 30,000 g/mol to about 2,500,000 g/mol.

4. The biomedical device of claim 1, wherein the polyethylene comprises a copolymer of ethylene and at least one monomer selected from the group consisting of propylene, butylene, and any combination thereof.

5. The biomedical device of claim 1, wherein the polyethylene is crosslinked.

6. The biomedical device of claim 1, wherein the polyethylene composition is foamed.

7. The biomedical device of claim 1, wherein the molded polyethylene component is nonload-bearing.

8. The biomedical device of claim 1, wherein the biomedical device is nonload-bearing.

9. The biomedical device of claim 1, wherein the biomedical device is load-bearing.

10. The biomedical device of claim 1, wherein the molded polyethylene component has been formed into a desired shape from a polyethylene composition melt that comprises the polyethylene.

11. The biomedical device of claim 1, wherein the polyethylene composition further comprises an active pharmaceutical ingredient.

12. The biomedical device of claim 1, wherein the polyethylene composition further comprises an imaging agent.

13. The biomedical device of claim 1, wherein the polyethylene composition further comprises at least one selected from the group consisting of a second polymer, a plasticizer, a compatibilizer, an antioxidant, a reinforcing filler, a pigment, a dye, a radioopaque filler, a lubricant, a processing aid, a light stabilizer, a neutralizer, an antiblock, and any combination thereof.

14. The biomedical device of claim 1, wherein the molded polyethylene component further comprises a reinforcing structure, wherein the polyethylene composition is disposed on at least a portion of a surface of the reinforcing structure.

15. The biomedical device of claim 14, wherein the reinforcing structure comprises at least one selected from the group consisting of ultra high molecular weight polyethylene, a metal, a metal alloy, a ceramic, and any combination thereof.

16. The biomedical device of claim 1, wherein the biomedical device further comprises other components, and wherein the molded polyethylene component substantially encases the other components.

17. The biomedical device of claim 1 being at least one selected from the group consisting of a chip, an RFID tag, tubing, a pump, a feeding tube, a catheter, a vascular catheter, a prosthetic, an inflatable balloon, a stent, a heart valve, a neurostimulator, a cochlear implant, a cranio-maxillofacial implant, synthetic cartilage, a stomach ring, a surgical instrument, a blood vessel clamp, an aneurysm clip, a spinal plug for use in conjunction with a joint fusion system, a base-plate stem cap for an artificial joint, a muscle implant, a nasopharyngeal implant, a laryngeal implant, a drug delivery device, a transdermal patch, a subdermal implant, an oromucosal insert, an intrauterine device, an intravaginal ring, and a dental fiber.

18. A kit comprising the biomedical device of claim 1 and a set of instructions.

19. A method comprising:
implanting the biomedical device of claim 1 into a patient.

20. A method comprising:
treating a patient with the biomedical device of claim 1.

21. A biomedical device comprising:
a conduit that comprise at least one wall defining an internal volume, the at least one wall comprising:
polyethylene having a melt flow index of about 3.5 g/10 min or greater as measured by ASTM D1238 at 190° C./21.5 kg weight and having an ash content of about 500 ppm or less.

22. The biomedical device of claim 21, wherein the polyethylene has a specific gravity of about 0.93 g/cm³ to about 0.97 g/cm³.

23. The biomedical device of claim 21, wherein the polyethylene has an average molecular weight of about 30,000 g/mol to about 2,500,000 g/mol.

24. The biomedical device of one claim 21, wherein the polyethylene is crosslinked.

25. The biomedical device of claim 21, wherein the polyethylene comprises a copolymer of ethylene and at least one monomer selected from the group consisting of propylene, butylene, and any combination thereof.

26. The biomedical device of claim 21 being at least one selected from the group consisting of a pump, tubing, a feeding tube, a catheter, a vascular catheter, a stent, a heart valve, a stomach ring, a surgical instrument, a nasopharyngeal implant, and a laryngeal implant, a drug delivery device, an intrauterine device, and an intravaginal ring.

27. The biomedical device of claim 21 further comprising:
at least one wire disposed within the internal volume.

28. The biomedical device of claim 27 being at least one selected from the group consisting of a pacemaker, a neurostimulator, and a cochlear implant.

29. A kit comprising the biomedical device of claim 21 and a set of instructions.

30. A method comprising:
implanting the biomedical device of claim 21 into a patient.

31. A method comprising:
treating a patient with the biomedical device of claim 21.

32. A method comprising:
providing a polyethylene composition melt that comprises polyethylene having a melt flow index of about 3.5 g/10 min or greater as measured by ASTM D1238 at 190° C./21.5 kg weight and having an ash content of about 500 ppm or less; and
extruding the polyethylene composition melt to form a molded polyethylene component of a biomedical device.

33. The method of claim 32, wherein the polyethylene composition melt further comprises an additive that comprises at least one selected from the group consisting of a plasticizer, a compatibilizer, an active pharmaceutical ingredient, a contrast agent, an antioxidant, a reinforcing filler, a pigment, a dye, a radioopaque filler, a lubricant, a processing aid, a light stabilizer, a neutralizer, an antiblock, and any combination thereof.

34. The method of claim 32, wherein the polyethylene composition melt further comprises at least one second polymer selected from the group consisting of a polyethylene, linear low density polyethylenes, low density polyethylenes, a polypropylene, a graft-modified olefin polymer, a chlorinated polyethylene, a thermoplastic vulcanizate, a polyether ether ketone, a polyisoprene, a polyester, a polyamide, an ethylene vinyl acetate copolymer, an ethylene vinyl-methacrylate copolymer, a silicone, a polyethylene glycol, poly lactic acid, poly glycolic acid, polyethylene imine, a polyurethane, polyacrylonitrile, a styrene block copolymer, a rubber, an ethylene-carboxylic acid copolymer, an ethylene acrylate copolymer, polybutylene, polybutadiene, a nylon, a polycarbonate, an ethylene ethylacrylate polymer, an ethylene styrene interpolymer, any derivative thereof, any copolymer thereof, and any combination thereof.

35. The method of one claim 32, wherein the polyethylene composition is extruded onto at least a portion of a surface of the reinforcing structure.

36. The method of claim 32 further comprising:
applying to the molded polyethylene component at least one selected from the group consisting of an active pharmaceutical ingredient, a contrast agent, an antioxidant, a pigment, a dye, a radioopaque filler, a lubricant, a processing aid, a light stabilizer, a neutralizer, an antiblock, and any combination thereof.

37. The method of claim 32 further comprising:
assembling the biomedical device comprising the molded polyethylene component.

38. The method of claim 32, wherein the biomedical device is at least one selected from the group consisting of a chip, an RFID tag, tubing, a pump, a feeding tube, a catheter, a vascular catheter, a prosthetic, an inflatable balloon, a stent, a heart valve, a neurostimulator, a cochlear implant, a cranio-maxillofacial implant, synthetic cartilage, a stomach ring, a surgical instrument, a blood vessel clamp, an aneurysm clip, a spinal plug for use in conjunction with a joint fusion system, a base-plate stem cap for an artificial joint, a muscle implant, a nasopharyngeal implant, a laryngeal implant, a drug delivery device, a transdermal patch, a subdermal implant, an oromucosal insert, an intrauterine device, an intravaginal ring, and a dental fiber.

39. A method comprising:
providing a polyethylene composition melt that comprises polyethylene having a melt flow index of about 3.5 g/10 min or greater as measured by ASTM D1238 at 190° C./21.5 kg weight and having an ash content of about 500 ppm or less; and
injection molding the polyethylene composition melt to form a molded polyethylene component of a biomedical device.

40. A method comprising:
providing a component of a biomedical device; and
substantially encasing the component in polyethylene having a melt flow index of about 3.5 g/10 min or greater as measured by ASTM D1238 at 190° C./21.5 kg weight and having an ash content of about 500 ppm or less.

41. The method of claim 40, wherein substantially encasing involves over molding.

42. The method of claim 40, wherein the component is a wire.

43. A method comprising:
polymerizing at least one olefin in the presence of a catalyst so as to form polyethylene having a melt flow index of about 3.5 g/10 min or greater as measured by ASTM D1238 at 190° C./21.5 kg weight, the at least one olefin comprising ethylene and optionally at least one selected from the group consisting of propylene, butylene, and any combination thereof; and
treating the polyethylene to yield a treated polyethylene having an ash content of about 500 ppm or less.

44. The method of claim 43 further comprising:
forming a molded polyethylene component of a biomedical device from the treated polyethylene.

45. The method of claim 43 wherein treating involves exposing the polyethylene concurrently or in series to at least one selected from the group consisting of steam, an inert solvent the catalyst is at least partially soluble in, an acid, acetone, water, a halogenated solvent, and any combination thereof.

46. The method of claim 45, wherein treating further involves increased temperature and/or decreased pressure.

47. A method comprising:
providing a sheet comprising a polyethylene composition that comprises polyethylene having a melt flow index of about 3.5 g/10 min or greater as measured by ASTM D1238 at 190° C./21.5 kg weight and having an ash content of about 500 ppm or less; and
thermoforming the sheet into a molded polyethylene component of a biomedical device.

48. A method comprising:
providing a sheet comprising a polyethylene composition that comprises polyethylene having a melt flow index of about 3.5 g/10 min or greater as measured by ASTM D1238 at 190° C./21.5 kg weight and having an ash content of about 500 ppm or less; and
shrink wrapping the sheet onto a portion of a biomedical device or component thereof.
